(19) United States
(12) Patent Application Publication
(10) Pub. No.: US 2012/0077730 A1
(43) Pub. Date: Mar. 29, 2012

(54) Method for in Vitro Molecular Evolution of Protein Function

(76) Inventors: Karin Haraldsson, Lund (SE); Christina Furebring, Lund (SE); Karin Barchan, Lund (SE)

(21) Appl. No.: 13/260,394

(22) PCT Filed: Mar. 31, 2010
(86) PCT No.: PCT/GB10/00621

§ 371 Date: Dec. 16, 2011

(30) Foreign Application Priority Data
Mar. 31, 2009 (GB) 0905503.9

(51) Int. Cl. A61K 38/02; C12P 21/00; A61K 31/7088; C12P 19/34

(52) U.S. Cl. 514/1.1; 435/91.2; 435/69.1; 514/44 R

(57) ABSTRACT

The invention provides a method for generating a polynucleotide sequence or population of sequences from parent polynucleotide sequences, the method comprising the steps of (a) providing a population of parent polynucleotide molecules, which population comprises plus and minus strands, (b) treating the population of parent polynucleotide molecules to generate a population of polynucleotide fragments thereof, (c) incubating the population of polynucleotide fragments under conditions which permit the formation of overlapping fragment pairs and (d) amplifying the overlapping fragment pairs using a polymerase to generate one or more product polynucleotide molecules which differ in sequence from the parent polynucleotide molecules, wherein step (d) is performed, at least in part, under conditions which favor the introduction of mutations into the one or more product polynucleotide molecules.

### FIELD OF INVENTION

The present invention relates to a method for in vitro molecular evolution of protein function.

### BACKGROUND

Protein function can be modified and improved in vitro by a variety of methods, including site directed mutagenesis (Alber et al., Nature, 5; 330(6143):41-46, 1987) combinatorial cloning (Huse at al., Science, 246:1275-1281, 1989; Marks et al., Biotechnology, 10: 779-783, 1992) and random mutagenesis combined with appropriate selection systems (Barbas et al., PNAS. USA, 89: 4457-4461, 1992).

The method of random mutagenesis together with selection has been used in a number of cases to improve protein function and two different strategies exist. Firstly, randomisation of the entire gene sequence in combination with the selection of a variant (mutant) protein with desired characteristics, followed by a new round of random mutagenesis and selection. This method can then be repeated until a protein variant is found which is considered optimal (Schier R. et al., J. Mol. Biol. 1996 263 (4): 551-567). Here, the traditional route to introduce mutations is by error prone PCR (Leung et al., Technique, 1: 11-15, 1989) with a mutation rate of approximately 0.7%. Secondly, defined regions of the gene can be mutagenised with degenerate primers, which allows for mutation rates of up to 100% (Griffiths et al., EMBO. J, 13: 3245-3260, 1994; Yang et al., J. Mol. Biol. 254: 392-403, 1995).

Random mutation has been used extensively in the field of antibody engineering. Antibody genes formed in vivo can be cloned in vitro (Larrick et al., Biochem. Biophys. Res. Commun. 160: 1250-1256, 1989) and random combinations of the genes encoding the variable heavy and light genes can be subjected to selection (Marks et al., Biotechnology, 10: 779-783, 1992). Functional antibody fragments selected by these methods can be further improved using random mutagenesis and additional rounds of selections (Schier R. et al., J. Mol. Biol. 1996 263 (4): 551-567).

Typically, the strategy of random mutagenesis is followed by selection. Variants with interesting characteristics can be selected and the mutated DNA regions from different variants, each with interesting characteristics, combined into one coding sequence (Yang et al., J. Mol. Biol. 254: 392-403, 1995).

Combinatorial pairing of genes has also been used to improve protein function, e.g. antibody affinity (Marks et al., Biotechnology, 10: 779-783, 1992).

Another known process for in vitro mutation of protein function, which is often referred to as “DNA shuffling”, utilises random fragmentation of DNA and assembly of fragments into a functional coding sequence (Stemmer, Nature 370: 389-391, 1994). The DNA shuffling process generates diversity by recombination, combining useful mutations from individual genes. It has been used successfully for artificial evolution of different proteins, e.g. enzymes and cytokines (Chang et al. Nature Biotech. 17, 793-797, 1999; Zhang et al. Proc. Natl. Acad. Sci. USA 94, 4504-4509,1997; Christians et al. Nature Biotech. 17, 259-264, 1999). The genes are randomly fragmented using DNase I and then reassembled by recombination with each other. The starting material can be either a single gene (first randomly mutated using error-prone PCR) or naturally occurring homologous sequences (so-called family shuffling).

DNase I hydrolyses DNA preferentially at sites adjacent to pyrimidine nucleotides, therefore it is a suitable choice for random fragmentation of DNA. However, the activity is dependent on Mg or Mn ions, Mg ions restrict the fragment size to 50 bp, while the Mn ions will give fragment sizes less than 50 bp. Therefore, in order to have all possible sizes for recombination the gene in question needs to be treated at least twice with DNase I in the presence of either of the two different ions, followed by removal of these very same ions.

Although, in theory, it is possible to shuffle DNA between any clones, if the resulting shuffled gene is to be functional with respect to expression and activity, the clones to be shuffled have preferably to be related or even identical, with the exception of a low level of random mutations. DNA shuffling between genetically different clones will generally produce non-functional genes.

The present invention seeks to provide improved methods for in vitro protein evolution. In particular, the invention aims to provide a method which allows ‘fine tuning’ of the degree of variation during the reassembly process. This is particularly useful where a relatively low number of desirable mutants are created during a first round of reassembly.

The method thus seeks to provide significant time savings for the in vitro protein evolution development process.

### SUMMARY OF INVENTION

According to a first aspect of the present invention, there is provided a method for generating a polynucleotide sequence or population of sequences from parent polynucleotide sequence, the method comprising the steps of:
(a) providing a population of parent polynucleotide molecules, which population comprises plus and minus strands;
(b) treating the population of parent polynucleotide molecules to generate a population of polynucleotide fragments thereof;
(c) incubating the population of polynucleotide fragments under conditions which permit the formation of overlapping fragment pairs; and
(d) amplifying the overlapping fragment pairs using a polymerase to generate one or more product polynucleotide molecules which differ in sequence from the parent polynucleotide molecules
   wherein step (d) is performed, at least in part, under conditions which favour the introduction of mutations into the one or more product polynucleotide molecules.

Step (a) comprises providing a population of parent polynucleotide molecules, which population comprises plus and minus strands.

In a preferred embodiment of the method of the invention, the parent polynucleotide molecules encode one or more protein motifs.

The parent polynucleotide molecules may be a nucleic acid, such as DNA, cDNA or RNA or a nucleic acid derivative such as PNA (Peptide Nucleic Acid), GNA (Glycol Nucleic Acid), or TNA (Threose Nucleic Acid). However, in a further embodiment of the method of the invention the parent polynucleotide molecules are preferably cDNA molecules.

In one embodiment, the parent polynucleotide molecules are double-stranded. However, in a preferred embodiment of the method of the invention, the parent polynucleotide molecules are single-stranded.

In another preferred embodiment, the population of parent polynucleotide molecules comprise a first sub-population and a second sub-population. Conveniently, the first population of polynucleotides consists of plus strands of parent polynucleotide molecules and second population of polynucleotides consists of minus strands of parent polynucleotide molecules. Alternatively, the first and/or second populations may comprise both plus and minus strands of parent polynucleotide molecules.

Step (b) comprises treating the population of parent polynucleotide molecules to generate a population of polynucleotide fragments thereof.

Suitable fragmentation methods are well known to those skilled in the art and include both enzyme-based methods and physical methods.

By controlling the parameters of the fragmentation treatment in step (b) the size of the polynucleotide fragments may be controlled. Determining the lengths of the polynucleotide fragments in this way avoids the necessity of having to provide a further step such as purifying the fragments of desired length from a gel.

Where first and second sub-populations of parent polynucleotide molecules are used, they may be fragmented separately.

In one embodiment, at least one parameter of the fragmentation treatment condition used for fragmentation of the first population of polynucleotide molecules is different from the equivalent parameter of the fragmentation treatment conditions used for fragmentation of the second population of polynucleotide molecules. By ‘equivalent parameter’ we mean the same parameter used in the fragmentation treatment conditions of the other population of single-stranded polynucleotide molecules.

For example, where fragmentation is achieved using an enzyme, suitable parameters which may be varied include enzyme type, enzyme concentration, reaction volume, duration of the digestion reaction, temperature of the reaction mixture, pH of the reaction mixture, length of parent polynucleotide sequences, the amount of parent polynucleotide molecules and the buffer composition of the reaction mixture.

The use of different parameters of the treatment conditions used for fragmentation of the first and second populations of polynucleotide molecules provides the advantage of increased variability in the variant polynucleotides produced by the method of the invention.

In a preferred embodiment, step (b) comprises exposing the parent polynucleotide molecules to one or more nucleases.

By ‘nuclease’ we mean a polypeptide, e.g. an enzyme or fragment thereof, having nucleolytic activity.

It will be appreciated by persons skilled in the art that any suitable nuclease may be used in digestion step (b) to generate polynucleotide fragments, for example exonucleases, endonucleases or restriction enzymes, or combinations thereof. Examples of such enzymes are well known in the art.

In one embodiment, the nuclease is an exonuclease. Thus, the exonucleolytic activity of the polypeptide is greater than the endonucleolytic activity of the polypeptide. More preferably, the polypeptide has exonucleolytic activity but is substantially free of endonucleolytic activity.

For example, the exonuclease may be selected from the group consisting of BAL31, exonuclease I, exonuclease V, exonuclease VII, exonuclease T7 gene 6, bacteriophage lambda exonuclease and exonuclease Rec J_{f}.

In an alternative embodiment, step (b) comprises exposing the parent polynucleotide molecules to a physical fragmentation stimulus. For example, the physical fragmentation stimulus may be selected from the group consisting of nebulisation, sonication, heat and hydrodynamic shearing.

Advantageously, step (b) comprises generation of fragments by random cleavage of the parent polynucleotide molecules. However, such random cleavage is not essential. Thus, in an alternative embodiment step (b) comprises generation of fragments by non-random cleavage of the parent polynucleotide molecules.

Step (c) comprises incubating the population of polynucleotide fragments under conditions which permit the formation of overlapping fragment pairs.

By “overlapping fragment pairs” we include fragment pairs comprising a single-stranded ‘plus’ strand and a single-stranded ‘minus’ strand, which strands are co-annealed in an anti-parallel orientation (i.e. 5′-3′ oriented strand annealed with 3′-5′ oriented strand), to form a pair of partially overlapping nucleic acid strands having sequence complementarity in the area of overlap. The orientation of overlapping strands is such that the free 5-ends are distal to the area of overlap. For example:

3′- G-A-T-T- C-G-A-A-T-5′
5′-A-G-T-C-C-T- A-A-G -3′

It will be appreciated by skilled persons that the degree of complementarity in the area of overlap need not be 100%; it simply needs to be sufficiently high to allow the formation of overlapping fragment pairs during the temperature cycles of a PCR reaction.

In one embodiment, step (c) comprises first incubating the population of polynucleotide fragments under conditions which permit denaturation of double-stranded fragments followed by incubating the population of polynucleotide fragments under conditions which permit re-annealing of single-stranded fragments to generate overlapping fragment pairs.

Step (d) comprises amplifying the overlapping fragment pairs using a polymerase to generate one or more product polynucleotide molecules which differ in sequence from the parent polynucleotide molecules.

It will be appreciated that step (d) comprises an initial assembly stage, in which the fragments are elongated to create full-length product polynucleotides, following by an amplification stage, in which the full-length product polynucleotides are PCR-amplified.

In one embodiment, step (d) comprises repeated cycles of:
(i) extending the polynucleotide strands of the overlapping fragments pairs to generate extended fragment pairs;
(ii) denaturation of the constituent strands of the extended fragment pairs; and
(iii) re-annealing of the constituent strands to generate new overlapping fragment pairs.

Step (d) may further comprise the addition of primers to permit or encourage amplification of full-length product polynucleotide molecules.

In a further embodiment, step (d) comprises PCR amplification of the one or more product polynucleotide molecules.

In another embodiment, step (d) comprises cloning the one or more product polynucleotide molecules into an expression vector.

A key feature of Step (d) is that it is performed, at least in part, under conditions which favour the introduction of mutations into the one or more product polynucleotide molecules. Such conditions may be employed in the initial assembly stage and/or in the subsequent amplification stage. In one embodiment, conditions which favour the introduction of mutations are employed in both the assembly and amplification stages.

Such use of conditions which favour the introduction of mutations during the latter assembly and/or amplification stages distinguishes the methods of the invention from those disclosed in the prior art, which typically rely upon shuffling of naturally-occurring sequence variation in the starting (parent) polynucleotides. In some prior art methods, error-prone PCR is utilized but this is only to generate sequence variation in the parent polynucleotides (i.e. in step ‘a’) or to create sequence variation in the digested fragments by amplifying them using error-prone PCR (i.e. in step ‘b’). However, the use of mutagenesis method such as using error-prone PCR during the latter assembly and/or amplification stages is neither known nor suggested in the prior art.

By “conditions which favour the introduction of mutations” we include any condition, treatment or state which favours the introduction of nucleotide sequence mutation during polymerase-driven strand extension. Thus, we include any conditions which provide sub-optimal fidelity during polymerase-driven strand extension (relative to standard PCR amplification, e.g. using Taq polymerase). For example, the conditions may be chosen to provide a mutation frequency for at least 10⁻⁵ er base pair per duplication, preferably at least 5×10⁻⁵ per base pair per duplication, 10⁻⁴ per base pair per duplication, 10⁻³ per base pair per duplication, 10⁻² per base pair per duplication or 10⁻¹ per base pair per duplication.

When it was first derived, nucleic acid polymerisation, for example, polymerase chain reaction (PCR) found use mostly for the accurate amplification of known DNA sequences. However, polymerisation-based gene manipulation has since become invaluable for the alteration of genetic information at the molecular level.

Error prone polymerisation introduces random copying errors by imposing imperfect, and thus mutagenic, or ‘sloppy’, reaction conditions (e.g. by adding Mn²⁺ or Mg²⁺ to the reaction mixture (Cadwell and Joyce, 1991; Leung et al., 1989)) or by using error prone (and typically, non-proofreading capable) nucleic acid polymerases. This method has proven useful both for generation of randomised libraries of nucleotide sequences, and also for the introduction of mutations during the expression and screening process in a mutagenesis step.

Thus, in a preferred embodiment of the method of the invention in step (d), the conditions which favour the introduction of mutations into the one or more product polynucleotide molecules permit error-prone polymerisation.

The primary function of a polymerase is the polymerization of new DNA or RNA using an existing DNA or RNA template in the processes of replication and transcription. DNA polymerases “read” a template DNA strand and uses it to synthesize a new, complementary strand. However, errors can occur during polymerization with some polymerases being more error prone than others.

Error correction (or ‘proofreading’) is a property of some, but not all, DNA polymerases. This process corrects mistakes in newly-synthesized DNA. When an incorrect base pair is recognized, DNA polymerase reverses its direction by one base pair of DNA. The 3′->5′ exonuclease activity of the enzyme allows the incorrect base pair to be excised (this activity is known as proofreading). Following base excision, the polymerase can re-insert the correct base and replication can continue.

In one embodiment of the invention, step (d) comprises the use of a thermostable, non-proofreading polymerase to introduce mutations into the one or more product polynucleotide molecules.

Preferably the polymerase is a DNA polymerase. Although any non-proofreading polymerase may be utilised, it is preferably selected from the group consisting of Taq polymerase, Thermus flavus DNA polymerase I, Thermus thermophilus HB-8 DNA polymerase I, Thermophilus ruber DNA polymerase I, Thermophilus brokianus DNA polymerase I, Thermophilus caldophilus GK14 DNA polymerase I, Thermophilus filoformis DNA polymerase I, Bacillus stearothermophilus DNA polymerase I, Bacillus caldotonex YT-G DNA polymerase I, Bacillus caldovelox YT-F DNA polymerase I and other eubacterial DNA polymerases.

Suitable polymerases for use in introducing mutations into the one or more product polynucleotide molecules are also available commercially, for example Mutazyme and Mutazyme II (from Stratagene).

Thus, in a preferred embodiment of the invention, step (d) comprises the use of error-prone PCR to introduce mutations into the one or more product polynucleotide molecules.

Advantageously, the error-prone polymerisation has an error rate of at least 0.01%, for example at least 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 2.0%, 2.5%, 3%, 3.5%, 4%, 4.5%, 5%, 10%, 20%, 30% or higher.

Most preferably, the error-prone polymerisation has an error rate of between 0.08% and 5%.

Alternatively or additionally, the conditions which favour the introduction of mutations into the one or more product polynucleotide molecules may comprise the use of a mutagenesis enhancer in the polymerisation reaction. By “mutagenesis enhancer” we mean any substance which brings about mutagenesis, increases the rate of mutagenesis or otherwise modifies the mutagenic process (e.g. to favour particular types of mutagenesis or to favour mutation in a particular product polypeptide region).

In one preferred embodiment the mutagenesis enhancer is Mn²⁺.

In an alternative embodiment, the mutagenesis enhancer is a strand-destabilising nucleotide analogue. For example, the strand-destabilising nucleotide analogue may be selected from the group consisting of dITTP, dUTP, 7-deaza-dGTP, 8-oxo-dGTP and N4-methyl-2′-deoxycytidine 5′-triphosphate.

Alternatively or additionally, the conditions which favour the introduction of mutations into the one or more product polynucleotide molecules may comprise altering the concentration of one or more components in the polymerisation reaction. In a preferred embodiment, the Mg²⁺ concentration of the polymerisation reaction is altered. For example, the Mg²⁺ concentration may be increased. Thus, in one embodiment, the Mg²⁺ concentration may be increased by at least 2-fold compared to that used in conventional PCR, for example, 3-fold, 4-fold, 4.1-fold, 4.2-fold, 4.3-fold, 4.4-fold, 4.5-fold, 4.6-fold, 4.7-fold, 4.8-fold, 4.9-fold, 5-fold, 6-fold, seven-fold, eight-fold, 9-fold or 10-fold.

In a further alternative or additional embodiment, the concentration of dNTP is altered in the polymerisation reaction. For example, the relative concentration of dATP to dTTP may be altered in the polymerisation reaction. Alternatively, the relative concentration of dGTP to dCTP may be altered in the polymerisation reaction. In one embodiment of the invention, the relative concentration(s) of dATP to dTTP and/or dGTP to dCTP are altered by at least 2-fold, for example 3-fold, 4-fold, 5-fold, 6-fold, 7-fold, 8-fold, 9-fold or 10-fold.

Alternatively or additionally, the conditions which favour the introduction of mutations into the one or more product polynucleotide molecules may comprise altering the temperature of the elongation step in the polymerisation reaction. For example, the temperature of the elongation step may be increased (e.g. by between 0.1 and 60° C., for example, between 1 and 50° C., 1 and 40° C., 1 and 30° C., 1 and 20° C., 1 and 10° C. or 1 and 5° C.).

The invention thus provides a method for generating variant forms of a parent polynucleotide sequence.

It will be appreciated that the method of the invention may be carried out on any polynucleotide which encodes a polypeptide product, including any proteins having binding or catalytic properties, e.g. antibodies or parts of antibodies, enzymes or receptors. Furthermore, any polynucleotide that has a function that may be altered, such as catalytic RNA, may be mutated in accordance with the present invention. It is preferable that the parent polynucleotide encoding one or more protein motif is at least 12 nucleotides in length, more preferably at least 20 nucleotides in length, even more preferably more than 50 nucleotides in length. Polynucleotides being at least 100 nucleotides in length or even at least 200 nucleotides in length may be used. Where parent polynucleotides are used that encode large proteins such as enzymes or antibodies, these may be many hundreds or thousands of bases in length. The present invention may be carried out on any size of parent polynucleotide.

Advantageously, the mutation introduced into the one or more product polynucleotide molecules in step (d) is associated with an altered property or characteristic of the encoded polypeptide.

The altered property or characteristic of a polynucleotide or polypeptide generated by the method of the invention may be any variation or alteration in the normal activity of the wild type (parent) polynucleotide or of the polypeptide, protein or protein motifs it encodes. For example, the methods of the invention may be applied as follows:
(i) to modulate, either positively or negatively, the catalytic activity of an enzyme;
(ii) to modulate the binding specificity and/or affinity of an antibody;
(iii) to modulate the binding specificity and/or affinity of a ligand-receptor interaction, e.g. between an interleukin and its receptor (by producing variants of the ligand and/or the receptor);
(iv) to modulate the ability of a polypeptide monomer to form multimeric formations, e.g. in virus coat proteins for vaccines;
(v) to modulate the ability of an immunogen to stimulate the production of specific antibodies against it; and
(vi) to modulate the stability of a protein (e.g. serum stability of hormones and growth factors).

Thus, it will be appreciated that the methods of the invention may be used to alter a property/function of any protein, polypeptide or polynucleotide.

One preferred embodiment of the method of the invention further comprises the step of expressing at least one of the product polynucleotide molecules generated in step (d) to produce the encoded polypeptide. In a further preferred embodiment, comprises the step of testing the encoded polypeptide for altered characteristics.

Methods for testing variant polynucleotides or polypeptides generated by the method of the invention for altered properties are well known in the art. For example, selection of functional proteins from molecular libraries has been revolutionised by the development of the phage display technology (Parmley at al., Gene, 73: 305-391 1988; McCafferty et al., Nature, 348: 552-554, 1990; Barbas et al., PNAS. USA, 88: 7978-7982, 1991). In this method, the phenotype (protein) is directly linked to its corresponding genotype (DNA) and this allows for direct cloning of the genetic material, which can then be subjected to further modifications in order to improve protein function. Phage display has been used to clone functional binders from a variety of molecular libraries with up to 10″ transformants in size (Griffiths at al., EMBO. J. 13: 3245-3260, 1994). Thus, phage display can be used to clone directly functional binders from molecular libraries, and can also be used to improve further the clones originally selected. Other types of viruses that have been used for surface expression of protein libraries and selections thereof are baculovirus (Boublik et al Biotechnol 13:1079-1084. 1995; Mottershead et al Biochem Biophys Res Com 238:717-722, 1997; Grabherr et al Biotechniques 22:730-735, 1997) and retrovirus (Buchholz et al Nature Biotechnol 16:951-954, 1998).

Selection of functional proteins from molecular libraries can also be performed by cell surface display. Also here, the phenotype is directly linked to its corresponding genotype. Bacterial cell surface display has been used for e.g. screening of improved variants of carboxymethyl cellulase (CMCase) (Kim et al Appl Environ Microbiol 66:788-93, 2000). Other cells that can be used for this purpose are yeast cells (Boder and Wittrup Nat. Biotechnol 15:553-557, 1997), COS cells (Higuchi at al J Immunol Meth 202:193-204, 1997) and insect cells (Granzerio at al J Immunol Meth 203:131-139, 1997; Ernst et al Nucleic Acids Res 26:1718-1723, 1998).

The parent polynucleotide preferably encodes one or more protein motifs. These are defined as regions or elements of polynucleotide sequence that encode a polypeptide (i.e. amino acid) sequence which has a characteristic protein function. For example, a protein motif may define a portion of a whole protein, such as an epitope, a cleavage site or a catalytic site etc.

Several searchable databases of protein motifs and potential protein motifs are available, such as MOTIF, PROSITE, SMART and BLOCKS (www.blocks.fhcrc.org).

In one particularly preferred embodiment of the first aspect of the invention there is provided a method of comprising the steps of:
(a) providing a first population of polynucleotide molecules and a second population of single-stranded polynucleotide molecules, the first and second populations together constituting plus and minus strands of one or more parent polynucleotide molecules;
(b) digesting the first and second populations of polynucleotide molecules with an exonuclease to generate single-stranded polynucleotide fragments;
(c) contacting said single-stranded polynucleotide fragments generated from the plus strands with single-stranded fragments generated from the minus strands (under conditions which permit annealing of fragments); and
(d) amplifying the fragments that anneal to each other to generate a library of product polynucleotide molecules, at least some of which differ in sequence from the parent polynucleotide molecules
   wherein step (d) comprises the use of error-prone PCR to introduce one or more mutations into the product polynucleotide molecules.

A second, related aspect of the invention provides polynucleotide sequences obtained or obtainable by the method described above having an altered nucleotide sequence (preferably encoding a polypeptide having altered/desired characteristics). These polynucleotide sequences may be used for generating gene therapy vectors and replication-defective gene therapy constructs or vaccination vectors for DNA-based vaccinations. In addition, the polynucleotide sequences may be used as research tools.

A third aspect of the invention provides a polynucleotide library of sequences generated by the method described above from which a polynucleotide may be selected which encodes a protein having the altered/desired characteristics.

It is preferable that the polynucleotide library is a DNA library, for example a cDNA library.

In order to obtain expression of the generated polynucleotide sequence, the polynucleotide may be incorporated in a vector having control sequences operably linked to the polynucleotide sequence to control its expression. The vectors may include other sequences such as promoters or enhancers to drive the expression of the inserted polynucleotide sequence, further polynucleotide sequences so that the protein encoded for by the polynucleotide is produced as a fusion and/or nucleic acid encoding secretion signals so that the protein produced in the host cell is secreted from the cell. The protein encoded for by the polynucleotide sequence can then be obtained by transforming the vectors into host cells in which the vector is functional, culturing the host cells so that the protein is produced and recovering the protein from the host cells or the surrounding medium. Prokaryotic and eukaryotic cells are used for this purpose in the art, including strains of E. coli, yeast, and eukaryotic cells such as COS or CHO cells. The choice of host cell can be used to control the properties of the protein expressed in those cells, e.g. controlling where the protein is deposited in the host cells or affecting properties such as its glycosylation.

The protein encoded by the polynucleotide sequence may be expressed by methods well known in the art. Conveniently, expression may be achieved by growing a host cell in culture, containing such a vector, under appropriate conditions which cause or allow expression of the protein.

Systems for cloning and expression of a protein in a variety of different host cells are well known. Suitable host cells include bacteria, eukaryotic cells such as mammalian and yeast, and baculovirus systems. Also, utilising the retrovirus system for cloning and expression is a good alternative, since this virus can be used together with a number of cell types. Mammalian cell lines available in the art for expression of a heterologous polypeptide include Chinese hamster ovary cells, HeLa cells, baby hamster kidney cells, COS cells and many others. A common, preferred bacterial host is E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 3rd edition, Sambrook and Russell, 2001, Cold Spring Harbor Laboratory Press. Many known techniques and protocols for manipulation of polynucleotide sequences, for example in preparation of polynucleotide constructs, mutagenesis, sequencing, introduction of DNA into cells and gene expression, and analysis of proteins, are described in detail in Current Protocols in Molecular Biology, Ausubel et al. eds., John Wiley & Sons, 1992.

The system can be used for the creation of DNA libraries comprising variable sequences which can be screened for the desired protein function in a number of ways. Enzyme function can be screened for with methods specific for the actual enzyme function e.g. CMCase activity, β-glucosidase activity and also thermostability. Furthermore, phage display and cell surface display may be used for screening for enzyme function (Crameri A. et al., Nature 1998 15; 391 (6664):288-291; Zhang J. H. et al., PNAS. USA 1997 94 (9): 4504-4509; Warren M. S. et al., Biochemistry 1996, 9; 35(27): 8855-8862; Kim et al., Appl Environ Microbiol 66:788-93, 2000) as well as for altered binding properties of e.g. antibodies (Griffith et al., EMBO J. 113: 3245-3260, 1994).

Thus, the present invention also provides proteins, such as enzymes, antibodies, and receptors, having characteristics different to that of the wild type produced by the method of the first aspect of the invention.

Such expressed proteins may be used individually or within a pharmaceutically acceptable carrier as vaccines or medicaments for therapy, for example, as immunogens, antigens or otherwise in obtaining specific antibodies. They may also be used as research tools.

A polypeptide provided by the present invention may be used in screening for molecules which affect or modulate its activity or function. Such molecules may be useful in a therapeutic (possibly including prophylactic) context.

The present invention further provides a method comprising, following the identification of the polynucleotide or polypeptide having desired characteristics by the method described above, the manufacture of that polypeptide or polynucleotide in whole or in part, optionally in conjunction with additional polypeptides or polynucleotides.

Thus, a further aspect of the invention provides a method for making a polypeptide having altered/desired properties, the method comprising the following steps:
(a) generating mutated forms of a parent polynucleotide using a method according to any one of claims 1 to 46;
(b) expressing the variant polynucleotides produced in step (a) to produce variant polypeptides;
(c) screening the variant polypeptides for altered properties; and
(d) selecting a polypeptide having altered properties from the variant polypeptides.

The invention further provides a polypeptide obtained by the above method.

Following the identification of a polynucleotide or polypeptide having altered/desired characteristics, these can then be manufactured to provide greater numbers by well-known techniques such as PCR, cloning and expression within a host cell.

The resulting polypeptides or polynucleotides may be used in the preparation of industrial enzymes, e.g. laundry detergent enzymes where an increased activity is preferred at lower temperatures. Alternatively, the manufactured polynucleotide or polypeptide may be used as a research tool, i.e. antibodies may be used in immunoassays, and polynucleotides may be used as hybridisation probes or primers. Alternatively, the resulting polypeptides or polynucleotides may be used in the preparation of medicaments for diagnostic use, pharmaceutical use, therapy etc. as discussed as follows.

The polypeptides or polynucleotides generated by the methods of the invention and identified as having altered characteristics can be formulated in pharmaceutical compositions. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

Thus, the invention further provides a polynucleotide or polypeptide produced by the methods of the invention for use in medicine and the use of a polynucleotide or polypeptide produced by the methods of the invention in the preparation of a medicament for use in the treatment, therapy and/or diagnosis of a disease.

Whether it is a polypeptide, e.g. an antibody or fragment thereof, an enzyme, a polynucleotide or nucleic acid molecule, identified following generation by the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector (a variant of the VDEPT technique i.e. the activating agent, e.g. an enzyme, is produced in a vector by expression from encoding DNA in a viral vector). The vector could be targeted to the specific cells to be treated, or it could contain regulatory elements which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. an enzyme, in a vector by expression from encoding DNA in a viral vector (see for example, EP-A-415731 and WO 90/07936).

A composition may be administered alone or in combination with other treatments, either simultaneously or sequentially dependent upon the condition to be treated.

As a further alternative, the polynucleotide identified as having desirable characteristics following generation by the method of the present invention could be used in a method of gene therapy, to treat a patient who is unable to synthesize the active polypeptide encoded by the polynucleotide or unable to synthesize it at the normal level, thereby providing the effect provided by the corresponding wild-type protein.

Vectors such as viral vectors have been used in the prior art to introduce polynucleotides into a wide variety of different target cells. Typically the vectors are exposed to the target cells so that transfection can take place in a sufficient proportion of the cells to provide a useful therapeutic or prophylactic effect from the expression of the desired polypeptide. The transfected nucleic acid may be permanently incorporated into the genome of each of the targeted tumour cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpes viruses, including HSV and EBV, and retroviruses. Many gene therapy protocols in the prior art have used disabled murine retroviruses.

As an alternative to the use of viral vectors other known methods of introducing nucleic acid into cells includes electroporation, calcium phosphate co-precipitation, mechanical techniques such as microinjection, transfer mediated by liposomes and direct DNA uptake and receptor-mediated DNA transfer.

As mentioned above, the aim of gene therapy using nucleic acid encoding a polypeptide, or an active portion thereof, is to increase the amount of the expression product of the nucleic acid in cells in which the level of the wild-type polypeptide is absent or present only at reduced levels. Such treatment may be therapeutic in the treatment of cells which are already cancerous or prophylactic in the treatment of individuals known through screening to have a susceptibility allele and hence a predisposition to, for example, cancer.

The present invention also provides a kit for generating a polynucleotide sequence or population of sequences of desired characteristics comprising reagents for ssDNA preparation, an exonuclease and components for carrying out a PCR technique, for example, thermostable DNA (nucleotides) and a stopping device, for example, EGTA.

As outlined above the present invention conveniently provides for the creation of mutated enzyme gene sequences and their random combination to functional enzymes having desirable characteristics. As an example of this aspect of the invention, the enzyme genes are mutated by error prone PCR which results in a mutation rate of approximately 0.7%. The resulting pool of mutated enzyme genes are then digested with an exonuclease, e.g. BAL31, and the reaction inhibited by the addition of EGTA or by heat inactivation at different time points, resulting in a set of DNA fragments of different sizes. These may then be subjected to PCR based reassembly as described above. The resulting reassembled DNA fragments are then cloned and a gene library constructed. Clones may then be selected from this library and sequenced.

A further application of this technology is the generation of a population of variable DNA sequences which can be used for further selections and analyses. Besides encoding larger proteins, e.g. antibody fragments and enzymes, the DNA may encode peptides where the molecules functional characteristics can be used for the design of different selection systems. Selection of recombined DNA sequences encoding peptides has previously been described (Fisch et al., PNAS. USA 1996 Jul. 23; 93 (15): 7761-7766). In addition, the variable DNA population can be used to produce a population of RNA molecules with e.g. catalytic activities. Vaish et al., (PNAS. USA 1998 Mar. 3; 95 (5): 2158-2162) demonstrated the design of functional systems for the selection of catalytic RNA and Eckstein F (Ciba Found. Symp. 1997; 209; 207-212) has outlined the applications of catalytic RNA by the specific introduction of catalytic RNA in cells. The system may be used to further search through the sequence space in the selection of functional peptides/molecules with catalytic activities based on recombined DNA sequences.

Aspects and embodiments of the present invention will now be illustrated, by way of example, with reference to the accompanying figures. Further aspects and embodiments will be apparent to those skilled in the art.

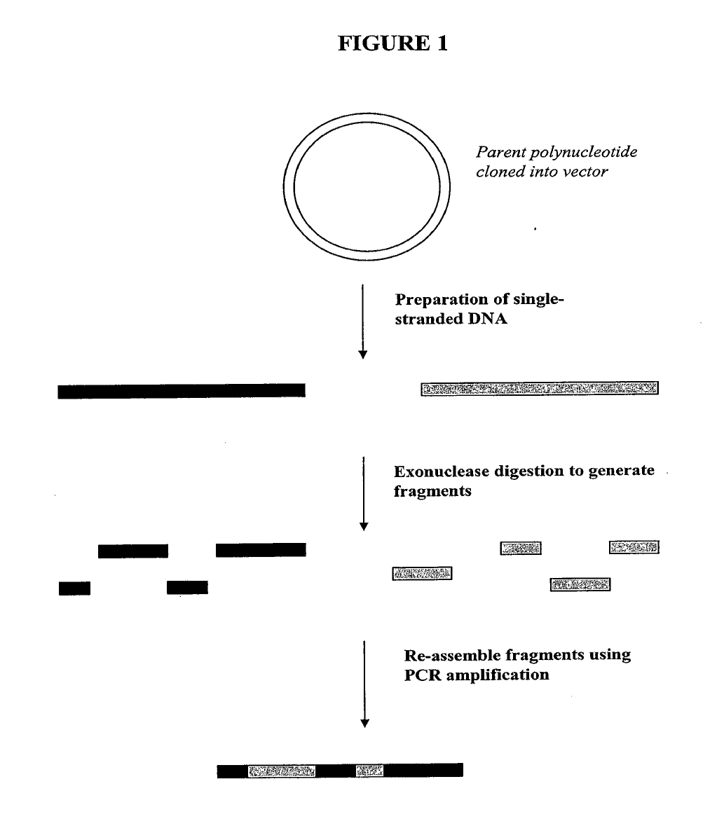

FIG. 1 shows the general principles of in vitro molecular evolution using the FIND™ technology of Alligator Bioscience (as described in WO 02/48351).

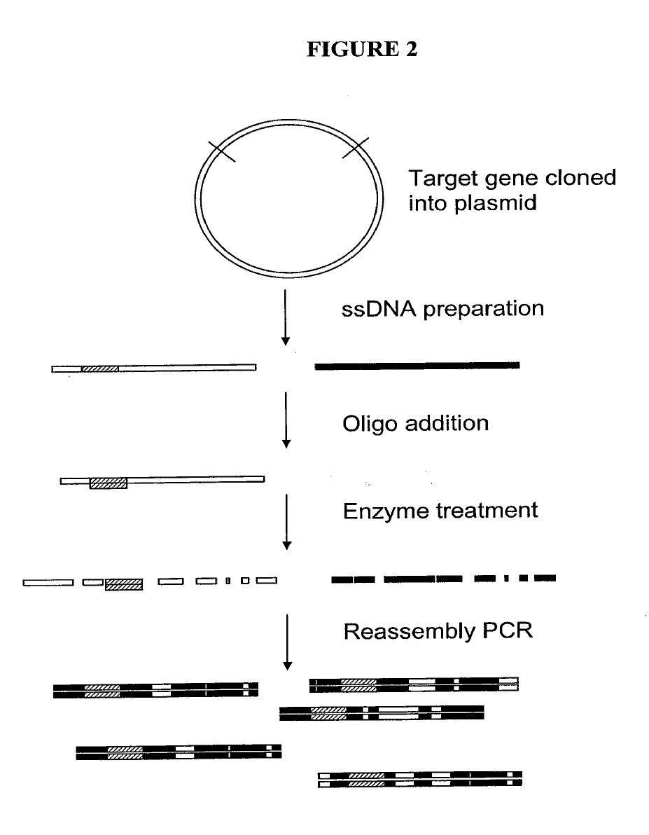

FIG. 2 shows a variation of the methods of the invention wherein the oligonucleotides of predetermined variability are added in step (b).

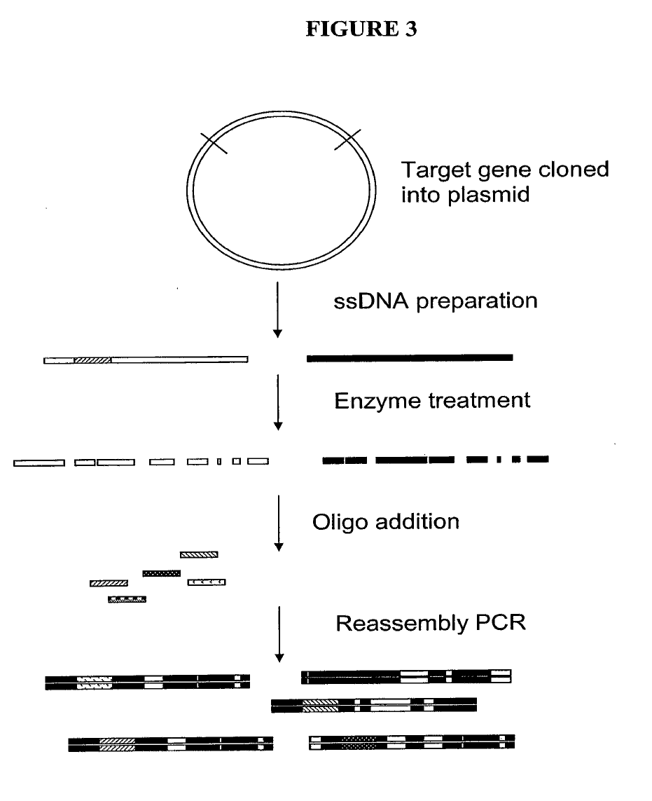

FIG. 3 shows a variation of the methods of the invention wherein the oligonucleotides of predetermined variability are added in step (c).

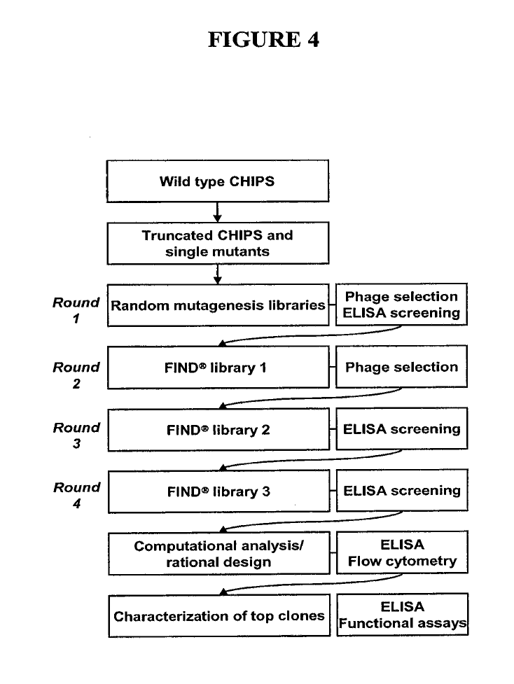

FIG. 4 shows the experimental strategy to decrease CHIPS interaction with human anti-CHIPS IgG, yet retaining C5aR blocking activity. An initial round of random mutagenesis and phage selection/ELISA screening was followed by three rounds of FIND® and phage selection/ELISA screening for decreased antibody binding and retained C5aR peptide binding. Then the structural distribution of the mutations in the improved clones was analyzed and new mutations were introduced by rational design. These clones were further analyzed for decreased antibody interaction and retained C5aR binding and inhibition.

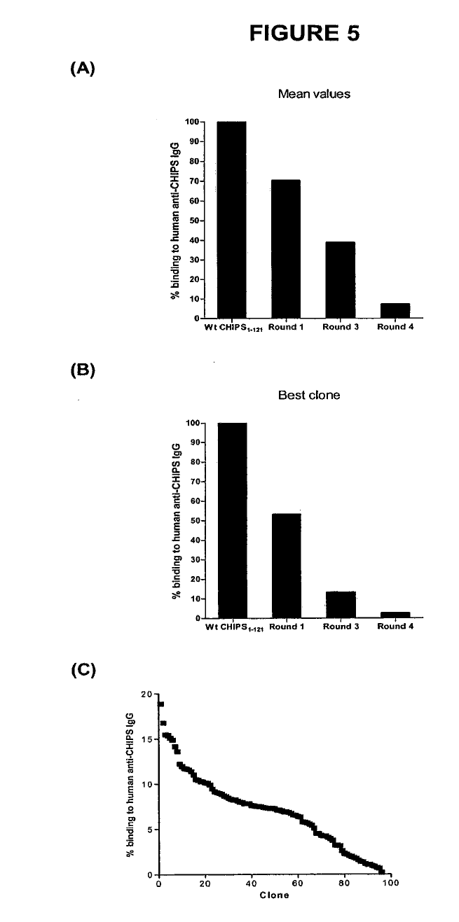

FIG. 5 shows a comparison of the mean values of clones (A) and best clones (B) from Round 1 (n=400), Round 3 (n=320) and Round 4 (n=96) to the wt CHIPS₁₋₁₂₁ as measured by % binding to human anti-CHIPS₃₁₋₁₁₃ IgG. The distribution of the 96 clones from Round 4 is shown in C.

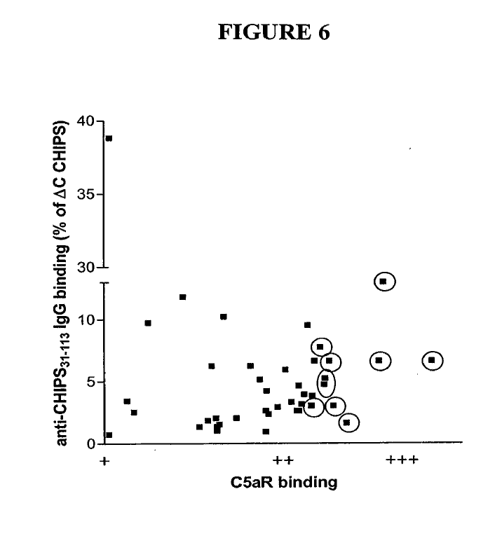

FIG. 6 shows a plot of the 42 best clones identified after the fourth round of diversification during the screening for decreased anti-CHIPS₃₁₋₁₁₃ IgG binding and retained C5aR binding. The 10 clones showing the highest binding to the human C5aR (in circles) were selected for further computational/rational design.

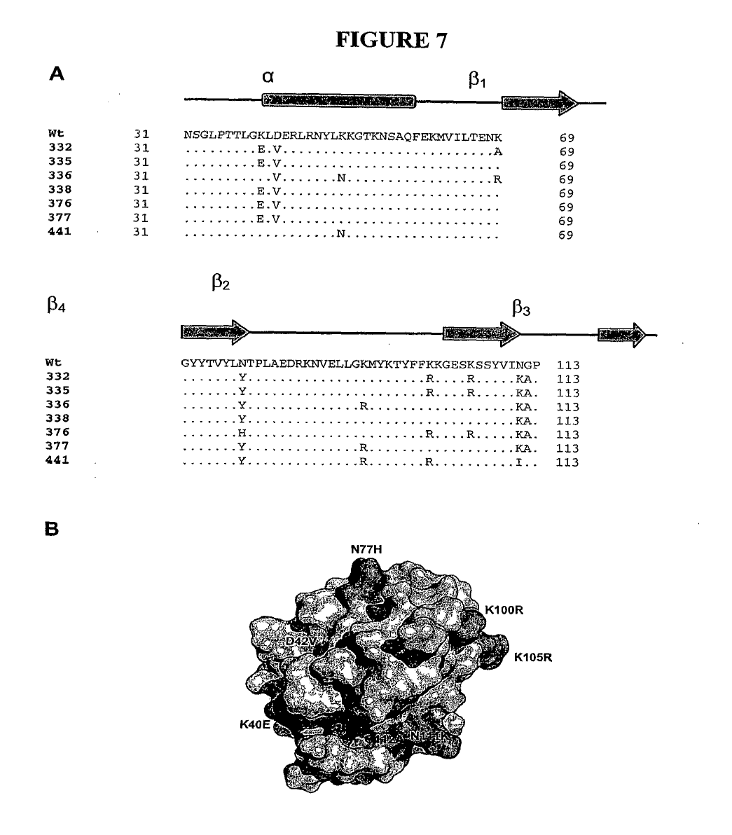

FIG. 7 shows the sequence alignment of the top seven clones after random mutagenesis, FIND® and rational design (A). Positions K40, D42, N77, N111 and G112 are mutated in almost all clones in different combinations with mutations in positions K50, K69, K92, K100 and K105. The mutated positions are positioned in the α-helix, in the loop between the β₁ and β₂ sheets, in the loop between the β₂ and β₃ sheets, in β sheet 3, in the loop between the β₃ and β₄ sheets and in β sheet 4. (B) Mutations in the selected clone (376) are marked in green in the CHIPS₃₁₋₁₁₃ structure modified from the CHIPS₃₁₋₁₂₁ structure (PDB code: 1×EE) (Haas et al., 2005). The figure was generated by the PyMOL molecular graphics system (DeLano, 2008).

### EXAMPLES

The methods of the present invention are particularly suited to use with the FIND® technology of Alligator Bioscience AB, Lund, Sweden.

Embodiments of the FIND® technology are shown schematically in FIGS. 1 to 3.

The FIND® technology is described in detail in WO 02/48351, WO 03/097834 and WO 2007/057682, the disclosure of which are incorporated herein by reference.

### Example 1

### The FIND® Technology

### Reagents

AmpliTaq® polymerase was purchased from Perkin-Elmer Corp., dNTPs from Boehringer Mannheim Biochemica (Mannheim, Germany), and BAL31 Nuclease from New England Biolabs Inc. (Beverly, USA). All restriction enzymes were purchased from New England Biolabs Inc. (Beverly, USA). Ethidium bromide was purchased from Bio-Rad Laboratories (Bio-Rad Laboratories, Hercules, Calif., USA). T4 DNA Ligase was purchased from New England Biolabs Inc. (Beverly, USA). EDTA and EGTA were purchased from Kebo Lab (Sweden).

All primers were designed in the laboratory and obtained from Life Technologies (Täby, Sweden) and SGS-DNA (Köping, Sweden).

### PCR

All Polymerase Chain Reactions (PCR) were carried out in an automatic thermocycler (Perkin-Elmer Cetus 480, Norwalk, Conn., and USA). PCR techniques for the amplification of nucleic acid are described in U.S. Pat. No. 4,683,195. References for the general use of PCR techniques include Mullis et al., Cold Spring Harbor Symp. Quant. Biol., 51:263, (1987), Ehrlich (ed), PCR technology, Stockton Press, NY, 1989, Ehrlich et al., Science, 252:1643-1650, (1991), “PCR protocols; A Guide to Methods and Applications”, Eds. Innis et al., Academic Press, New York, (1990).

### Sequencing

All constructs have been sequenced by the use of BigDye Terminator Cycle Sequencing kit (Perkin-Elmer, Elmervill, Calif., USA). The sequencing was performed on an ABI Prism 377 DNA Sequencer.

### Agarose Electrophoresis

Agarose electrophoresis of DNA was performed with 2% agarose gels (AGAROSE (FMC Bioproducts, Rockland, Me., USA)) with 0.25 μg/ml ethidium bromide in Tris-acetate buffer (TAE-buffer 0.04M Tris-acetate, 0.001M EDTA). Samples for electrophoresis were mixed with a sterile filtrated loading buffer composed of 25% Ficoll and Bromphenolic blue and loaded into wells in a the 2% agarose gel. The electrophoresis was run at 90 V for 45 minutes unless otherwise stated in Tris-acetate buffer with 0.25 μg/ml ethidium bromide. Bands of appropriate size were gel-purified using the Qiaquick Gel Extraction Kit (Qiagen GmbH, Hilden, Germany) when needed. As molecular weight standard, DNA molecular weight marker 1 kb ladder (Gibco BRL) was used. The DNA-concentration of the gel-extracted products was estimated using a spectrophotometer.

### Bacterial Strains

The Escherichia coli-strain TOP10F′ was used as a bacterial host for transformations. Chemically competent cells of this strain were produced basically as described Hanahan, D. 1983. Studies on transformation of Escherichia coli with plasmids. J. Mol. Biol. 166: 557-580. Electrocompetent cells of this bacterial strain were produced (Dower, W. J., J. F. Miller & C. W. Ragsdale. 1988: High efficiency transformation of E. coli by high voltage electroporation. Nucleic Acids Res. 16:6127).

### Plasmids

All genetic manipulations were performed in pFab5chis as described in Sambrook, Molecular cloning; a laboratory manual (Second Edition, Cold Spring Harbor Laboratory Press, 1989). The pFab5chis vector is designed to harbour any scFv gene inserted between SfiI and NotI sites (see Emgberg at al., 1995, Methods Mol. Biol. 51:355-376). The SfiI site is located in the peIB leader and the NotI site is located just after the VL region, such that VH-linker-VL is inserted. In this case, an antibody directed to CD40 was used.

### Primers

Two biotinylated primers surrounding the antibody gene of pFab5chis were designed with the following sequences including designated unique restriction sites:

1736 SfiI forward primer:
[SEQ ID NO: 1]
5′-ATT ACT CGC GGC CCA GC ▾ C GGC CAT GGC CCA CAG GTC AAG CTC GA
and
1735 NotI reversed primer:
[SEQ ID NO: 2]
5′-TTA GAG CCT GC ▾ G GCC GCC TTG TCA TCG TCG TCC TT
(wherein ‘▾’ designates the cleavage site)

Two non-biotinylated primers surrounding the antibody gene of pFab5chis were designed with the following sequences including designated restriction sites:

1664 SfiI forward primer:
[SEQ ID NO: 3]
5′-ATT ACT CGC GGC CCA GC ▾ C GGC CAT GGC CCA CAG GTC AAG CTC GA
and
[SEQ ID NO: 4]
1635 NotI reversed primer:
5′-TTA GAG CCT GC ▾ G GCC GCC TTG TCA TCG TCG TCC TT

### Standard PCR

Standard PCR reactions were run at 25 cycles consisting of following profile: denaturation (94° C., 1 minute), primer annealing (55° C., 1 minute) and extension (72° C., 3 minutes). Each PCR reaction contained 10 mM Tris-HCl, pH 8.3, 50 mM KCl, 1.5 mM MgCl₂, 200 μM dNTP, 1 μM forward primer, 1 μM reverse primer, 1.25 U AmpliTaq® thermostable DNA polymerase (Perkin-Elmer Corp.), and 50 ng template in a final volume of 100 μl.

### Error Prone PCR

The error prone PCR reactions were carried out in a 10× buffer containing 500 mM NaCl, 100 mM Tris-HCl, pH 8.8, 5 mM MgCl₂ 100 μg gelatine (according to Kuipers et al., Nucleic Acids Res. 1991, Aug. 25; 19 (16):4558 but with MgCl₂ concentration increased from 2 mM to 5 mM).

For each 100 μl reaction the following was mixed:

| | | |
|---|---|---|
| dATP 5 mM | 5 | μl |
| dGTP 5 mM | 5 | μl |
| dTTP 10 mM | 10 | μl |
| dCTP 10 mM | 10 | μl |
| 20 μM 3′ primer | 1.5 | μl |
| 20 μM 5′-primer | 1.5 | μl |
| 10x Kuipers buffer | 10 | μl |
| sterile mp H₂0 | 46.3 | μl |

The template in pFab5chis vector was added at an amount of 50 ng. 10 μl of 10 mM MnCl₂ was added and the tube was checked that no precipitation of MnO₂ occurred. At last 5 Units of Taq enzyme was added. The error prone PCR was run at the following temperatures for 25 cycles without a hot start: 94° C. 1′, 45° C. 1′, 72° C. 1′, +72° C. for 7 minutes. The resulting product was an error proned (i.e. mutated) insert of 750 bp. This insert was purified with Gibco PCR purification kit, before further treatment.

### Generation of Single-Stranded DNA by Biotinylated Primers

The fragment of interest was amplified by two separate PCR reactions. These reactions can be standard PCR as described above or error prone PCR also as described above. The primers should be designed so that in one reaction the forward primer is biotinylated and in the other reaction the reverse primer is biotinylated. For example, PCR reactions with A) primers 1736 and 1635 and B) primers 1664 and 1735, with the above mentioned profile was performed for 25 cycles with pFab5chis-antibody as template. This yielded PCR-products of approximately 750 bp: in A the upper strand was biotinylated; and in B the lower strand was biotinylated.

The non-biotinylated strands were retrieved by purification using a solid matrix coated with streptavidin e.g. Dynabeads. The magnetic beads are washed and equilibrated with PBS/1% BSA and B&W buffer containing 5 mM Tris pH 7.5, 1 M NaCl, and 0.5 mM EGTA. 100 μl of each PCR product is mixed with 100 μl beads dissolved in 2× B&W buffer and incubated at room temperature for 15 minutes with rotation. Unbound PCR products are removed by careful washing twice with B&W. The non-biotinylated strand of the captured DNA is eluted by alkaline denaturation by letting the DNA incubate with 25 μl 0.1 M NaOH for 10 minutes in room temperature. The solution is separated from the beads and neutralised with 7.5 μl 0.33 M HCl and 2.5 μl 1 M Tris pH 8.

### Generation of Single-Stranded DNA Using Phage

The fragment of interest was cloned into bacteriophage M13 vectors M13 mp 18 and M13 mp 19 using PstI/HindIII restriction enzymes. The bacteriophage were propagated using Escherichia coli-strain TOP10F′ according to conventional methods. Single-stranded DNA for the upper strand was prepared from bacteriophage vector M13 mp 18 and single-stranded DNA for the lower strand was prepared from bacteriophage vector M13 mp 19. Briefly, 1.5 ml of an infected bacterial culture was centrifuged at 12 000 g for 5 minutes at 4° C. The supernatant was precipitated with 200 μl 20% PEG8000/2.5 M NaCl. The pelleted bacteriophage was resuspended in 100 μl TE. 50 μl phenol equilibrated with Tris-Cl (pH 8.0) was added and the sample was vortexed. After centrifugation at 12 000 g for 1 minute at RT the upper phase, containing the DNA, was transferred and precipitated with ethanol. The DNA pellet was dissolved in 50 μl TE (pH 8.0) and stored at −20° C. (Sambrook et al. Molecular Cloning, A laboratory manual 2^{nd} edition. Cold Spring Habor Laboratory Press. 1989, chapter 4). Single-stranded DNA prepared from phage is circular and must be opened prior to BAL31 treatment. This can be performed with an endonuclease able to cleave single-stranded DNA.

### Generation of Single-Stranded DNA Using Asymmetric PCR

PCR products are purified using a spin column to remove excess primers from the previous PCR. 150 ng of the purified product is used as template in a linear amplification carried out in 100 μl of 1× GeneAmp® 10×PCR buffer containing 1.5 mM MgCl2 (Applied Biosystems), 200 μM of each dNTP (New England BioLabs), 1.25 U AmpliTaq® DNA Polymerase (Applied Biosystems) and 1.0 μM of a single primer. PCR cycle conditions are: denaturation at 94° C. for 1 minute, 35 cycles of 94° C. for 30 seconds, 55° C. for 30 seconds, 72° C. for 1 minute followed by extension at 72° C. for 7 minutes.

Asymmetric PCR products are size separated from double stranded template on a 1% agarose gel and purified using Qiaquick Gel Extraction Kit (Qiagen).

### Generation of Single-Stranded DNA Using Lambda Exonuclease

Initially a dsDNA fragment is produced using standard PCR reactions creating a DNA with unique restriction enzyme (RE) sites in the 5′ and 3′-end respectively. The PCR reaction is divided in two and RE digested respectively to create a 5′ phosphorylation preferentially with restriction enzymes creating 3′ overhang or blunt ends. The digestion is performed in suitable buffer and over night to accomplish complete digestion. If an enzyme creating a 5′ overhang has to be used the overhang can be filled in using a DNA polymerase. After purification 1-4 μg dsDNA is treated with 10U of Lambda exonuclease (eg Strandase™ from Novagen or Lambda exonuclease from NEB) in accompanied specific buffer for 30 min at 37° C. and the reaction is stopped at 75° C. for 10 min. The ssDNA is further separated from any dsDNA residues on an agarose gel using standard gel extraction methods.

### Generation of Single-Stranded Fragmented DNA Using BAL 31

The ssDNA strands (containing upper and lower strands, respectively) were subjected to separate enzymatic treatment using e.g. BAL 31 (i.e. upper strands were digested separately from lower strands). Each digestion reaction contained 0.02 □g/□l ssDNA, 600 mM NaCl, 20 mM Tris-HCl, 12 mM CaCl₂, 12 mM MgCl₂, 1 mM EDTA pH 8.0 and BAL 31 at various enzyme concentrations ranging from 0.1-5 U/ml. The reactions were incubated at 30° C. and fractions of digested ssDNA were collected sequentially at 10, 30, 60 and 120 seconds or longer. The reactions were stopped by addition of EDTA and heat treatment at 65° C. for 10 minutes. The ssDNA fragments were purified by phenol/chloroform extraction and ethanol precipitated. The ssDNA are resuspended in 10 mM Tris pH 8.0.

The digestion pattern was evaluated by 1% agarose gel electrophoresis.

### Purification of Digestion Produced Fragments:

Digested DNA fragments were purified by phenol/chloroform/isoamylalcohol extraction. 50 μl of buffered phenol was added to each tube of 100 μl sample together with 50 μl of a mixture of chloroform and isoamylalcohol (24:1). The tubes were vortexed for 30 seconds and then centrifuged for 1 minute in a microfuge at 14000 r.p.m. The upper phase was then collected and mixed with 2.5 volumes of 99.5% Ethanol ( 1/10 was 3M Sodium Acetate, pH 5.2). The DNA was precipitated for 1 hour in −80° C. The DNA was then pelleted by centrifugation for 30 minutes in a microfuge at 14.000 r.p.m. The pellet was washed once with 70% ethanol and then re-dissolved in 10 μl of sterile water.

### Analysis of Digestion Produced Purified Fragments on Agarose Gel

5 μl of the dissolved pellet from each time point and from the blank were mixed with 2.5 μl of loading buffer (25% Ficoll and Bromphenolic blue) and loaded into wells in a 2% agarose gel. The electrophoresis of the different time points was performed as above.

### Reassembly of Full Length Fragments

Reassembly of the ssDNA fragments is achieved by two sequential PCR reactions. The first PCR reaction should contain 10 mM Tris-HCl, pH 8.3, 50 mM KCl, 1.5 mM MgCl₂, 200 μM dNTP, 0.3 U Taq polymerase and 2 μl BAL31 treated sample, all in a final volume of 25 μl, and subjected to 5 cycles with the following profile: 94° C. for 1 minute, 50° C. for 1 minute and 72° C. for 2 minutes+72° C. for 5 minutes. The second PCR reaction should contain 10 mM Tris-HCl, pH 8.3, 50 mM KCl, 1.5 mM MgCl₂, 200 μM dNTP, 0.6 U Taq polymerase, 1 μM forward primer, 1 μM reverse primer, and 5 μl sample from the first PCR reaction, all in a final volume of 50 μl, and subjected to 15 cycles with the following profile: 94° C. for 1 minute, 55° C. for 1 minute and 72° C. for 2 minutes+72° C. for 7 minutes.

The resulting products can be evaluated by agarose gel electrophoresis.

Restriction Digestion of Reassembled Fragment and Plasmid with SfiI and NotI

The reassembled fragment and the plasmid pFab5chis were first cleaved with SfiI by using NEB buffer 2 including BSA and 11 U enzyme/μg DNA. The reaction was carried out for 4 h at 50° C. After this the DNA was cleaved with NotI by adding conversion buffer and 6 U enzyme/μg DNA. This reaction was carried out for 37° C. overnight.

### Gel Purification of Restriction Digested Vector and Restriction Digested Reassembled Fragment

The cleavage reactions were analysed on a 1% agarose gel. The restriction digested insert showed a cleavage product of about 750 bp. This corresponds well with the expected size. The band of the cleaved insert and plasmid was cut out and gel-extracted as previously described.

Ligation of Reassembled Restriction Digested Fragment with Restriction Digested pFab5chis

Purified cleaved pFab5chis was ligated with purified reassembled restriction digested fragment at 12° C. water bath for 16 hours. 50 μl of the vector was mixed with 50 μl of the insert and 15 μl of 10× buffer (supplied with the enzyme), 7.5 μl ligase (5 U/μl) and sterile water to a final volume of 150 μl. A ligation of restriction digested pFab5chis without any insert was also performed in the same manner.

Transformation of Chemically Competent E Coli TOP10F′ with the Ligated Reassembled Insert and pFab5chis

The ligation reactions were purified by phenol/chloroform extraction as described above. The upper phase from the extraction was collected and mixed with 2.5 volumes of 99.5% Ethanol ( 1/10 was 3M Sodium Acetate, pH 5.2). The DNA was precipitated for 1 hour in −80° C. The DNA was then pelleted by centrifugation for 30 minutes in a microfuge at 14.000 r.p.m. The pellet was washed once with 70% ethanol and then re-dissolved in 10 μl of sterile water. 5 μl of each ligation was separately mixed with 95 μl chemically competent E coli TOP10F incubated on ice for 1 hour and then transformed (Sambrook et al. Molecular Cloning, A laboratory manual 2^{nd} edition. Cold Spring Habor Laboratory Press, 1989). After one hour's growth the bacteria from the two transformations were spread onto ampicillin containing agar plates (100 μg/ml). The plates were grown upside-down in a 37° C. incubator for 14 hours.

### Example 2

### Directed Evolution of CHIPS to Generate Functional Variants with Reduced Interaction with Human Antibodies

### Introduction

Inflammation is the tissue response to injury or infection by pathogens. The attraction of immune cells and soluble molecules to the site of damage or infection initiates the healing process. Even though the ability to raise an inflammatory response is crucial for survival, the ability to control inflammation is also necessary for health. Anti-inflammatory drugs aim at blocking key events in inflammation for treatment of disorders with excessive or uncontrolled inflammation. Examples of such drugs are Remicade® and Kineret®, approved for treatment of rheumatoid arthritis.

Many bacteria have evolved strategies to evade the human immune system, for example by avoiding recognition, or by secreting proteins that neutralize the antibacterial effects mediated by the immune system. Chemotaxis Inhibitory Protein of Staphylococcus aureus (CHIPS) is a 14.1 kDa protein which is a potent inhibitor of immune cell recruitment and activation associated with inflammation, through binding and blocking the C5a receptor (C5aR) and the formylated peptide receptor (De Haas et al., 2004; Postma et al., 2004). This way, CHIPS is a promising anti-inflammatory protein for treatment of several inflammatory diseases, e.g. sepsis (Rittirsch et al., 2008) or ischemia-reperfusion injury and immune complex disease (Heller et al., 1999) However, most individuals have pre-formed titers of antibodies specific for CHIPS (Wright et al., 2007). These antibodies might neutralize the function of CHIPS or induce an immune reaction, hence the CHIPS molecule would benefit from optimization to function well as a drug in the human circulation.

Directed evolution is an established approach for improving proteins. It has been utilized to improve many protein functions such as stability, activity or affinity (Johannes et al., 2006). Importantly for the development of protein therapeutics, directed evolution has proven to be a useful tool for generating protein variants with enhanced therapeutic potential (Yuan et al., 2005). The directed evolution approach is particularly efficient as it does not require prior knowledge of the structure of the protein. Instead of using inefficient and time consuming methods based on site-directed mutagenesis, rounds of gene recombination and high-throughput screening can be performed to identify improved variants. The process can be repeated and beneficial mutations will be accumulated while mutations not required for the property of interest will be excluded, as reviewed by (Yuan et al., 2005) and (Zhao, 2007).

Several distinct methods for directed evolution have been described in the literature; among them DNA shuffling (Stemmer, 1994a; Stemmer, 1994b) and the Staggered Extension process (StEP) (Yuan et al., 2005; Zhao et al., 1998). Another DNA recombination technology called Fragment INduced Diversity (FIND®), has previously proven to be useful in the optimization of thermostability of carboxypeptidase U (Knecht et al., 2006) and the activity of IL-1 receptor antagonists (Dahlen et al., 2008).

Even though directed evolution has been successfully utilized to identify new and improved protein variants, a limitation with this type of technology is the incapability of screening the entire sequence space of a protein. However, sequence space can be explored more efficiently if directed evolution is combined with computational tools and rational design (Wong et al., 2007; Zhao, 2007).

In this example, FIND® recombination was used in combination with rational/computational design of the CHIPS gene with the aim to create new protein variants with lower interaction with specific human antibodies. An improved CHIPS molecule would be characterized by decreased reactivity with pre-existing antibodies, but also preserved activity towards the C5aR. Therefore, receptor binding was monitored in parallel with the screening process for decreased antibody interaction. This way, we were able to isolate new CHIPS variants with significantly reduced interaction with human anti-CHIPS antibodies yet preserved C5aR blocking activity.

### Materials & Methods

### Cloning, Expression and Purification of Recombinant Proteins

Wild-type (Wt) CHIPS₁₋₁₂₁ was cloned, expressed and purified as described earlier (De Haas et al., 2004). CHIPS with truncated C-terminus (CHIPS ΔC) was 112 amino acids long with two additional non-relevant amino acids included in the C-terminal end of the expressed protein as a result of cloning (CHIPS₁₋₁₁₂). Genes encoding CHIPS ΔC and its corresponding single mutants K61A, K69A and K100A as well as CHIPS ΔN/C(CHIPS₃₁₋₁₁₃) were created from the gene encoding wt full-length CHIPS₁₋₁₂₁ by truncation and site-directed mutagenesis. These CHIPS variants were then cloned and expressed as described above. Single mutants were used for structural analysis by Haas et al. (Haas et al., 2005), but were also screened for anti-CHIPS IgG binding and mutants K61A, K69A and K100A showed decreased binding (data not shown).

CHIPS variants selected from libraries in this study were expressed in the same way, but purified from inclusion bodies (Gustafsson et al., 2009) or expressed by the Expressway Cell-Free E. coli Expression System (Invitrogen, Carlsbad, Calif.) as recommended by the manufacturer.

### Library Construction

### Random Mutagenesis

To create diverse libraries of CHIPS variants, two different methods of random mutagenesis were used to create in total four libraries. Error-prone PCR was performed as described previously (Leung et al., 1989). One library with high mutation frequency (library 1.1) and one with low mutation frequency (library 1.2) were created. A 20 cycle PCR was performed using primers (Fw: 5′-TCGCGGCCCAGCCGGCCATGGCCTTTACTTTTGAACCG-3′ [SEQ ID NO: 5] and Rev: 5′-GCCTGCGGCCGCAGATCTACCATTAATTACATAAG-3′ [SEQ ID NO: 6]) in the presence of 7.5 mM MgCl₂ and 0.64 mM MnCl₂. 2.5.0 AmpliTaq Thermostable DNA polymerase (Applied Biosystems, Foster City, Calif.) was added and the reaction was performed using the program 94° C., 5 min/(94° C., 30 s/55° C., 30 s/72° C., 40 s) 20 times and finally elongation at 72° C. for 10 minutes. GeneMorph II (Stratagene, La Jolla, Calif.) was used as recommended by the manufacturer. 10 pg DNA (a mixture of CHIPS ΔC and the corresponding K61A, K69A and K100A single mutants) was used for the design of the low mutation frequency library (library 1.4) and 1 ng DNA for the library with higher mutation frequency (library 1.3). The PCR reaction contained the primers described above and the PCR program was 95° C., 2 min/(95° C., 1 min/60° C., 1 min and 72° C., 1 min) 40 times and finally elongation at 72° C. for 10 minutes. To increase the mutation frequency in the 1 ng library, it was subjected to one more round of Genemorph II mutagenesis. This time, the amount of DNA in the PCR reaction was 10 ng. After purification, the PCR products were sub-cloned into the pGEM-T vector (Promega, Madison, Wis.) according to the manufacturer's recommendations and the sequences were analyzed and base exchanges evaluated

### FIND®

FIND® recombinations were performed as described in e.g. patents EP 1 341 909 and EP 1 504 098. Briefly, single stranded DNA was prepared by generating PCR products using one biotinylated and one regular primer. The PCR product was immobilized on a column containing streptavidin-conjugated magnetic beads (Miltenyi Biotec GmbH, Bergisch Gladbach, Germany) and placed in the magnetic field of a μMACS separator. The PCR product was denatured with 0.1 M NaOH and the eluted non-biotinylated DNA strand was collected and purified by agarose gel electrophoresis using Recochips (Takara Bio Inc., Shiga, Japan) according to the manufacturer's recommendations.

The FIND® experiments were initiated by fragmenting sense and antisense ssDNA, respectively, with Exonuclease I (Exo I) (New England Biolabs, Ipswich, Mass.) (100 U/μg DNA) for 10 minutes, Exonuclease V (Exo V) (USB, Cleveland, Ohio) (25 U/μg DNA) for 45 minutes and Exonuclease VII (Exo VII) (USB) (10 U/μg DNA) for 30 minutes in separate tubes in buffers as recommended by the manufacturers. The ssDNA fragments resulting from the exonuclease digestions were recombined in a PCR like reaction, without added primers, followed by amplification in a standard PCR reaction. After purification, PCR products were subcloned into the pGEM-T vector (Promega) according to the manufacturer's recommendations and sequences were analyzed.

### Protein Expression in Plate Format

CHIPS libraries created by random mutagenesis or FIND® recombinations were cloned into a modified pRSET B vector (Invitrogen) in BbsI and Bg/II sites for expression in E. coli. Libraries were transformed into E. coli BL21 star DE3 pLysS (Invitrogen), plated on 20 cm Qtray plates with LB agar supplemented with 50 μg/ml ampicillin and 34 μg/ml chloramphenicol and incubated at 37° C. overnight. The following day, E. coli colonies were picked and inoculated in 96 well round bottom plates containing 150 μl Luria Broth (LB) supplemented with 50 μg/ml ampicillin and 34 μg/ml chloramphenicol using a colony picker robot. The cultures were incubated at 37° C. with 78% humidity and shaking at 700 rpm in a Multitron plate shaker (Infors HT, Bottmingen, Switzerland) overnight. Day cultures were prepared from the overnight cultures by 1/100 dilution into fresh medium with 50 μg/ml ampicillin and incubation was continued at 37° C. as above. To induce protein expression, 0.5 mM IPTG (isopropyl β-D-thiogalactoside) was added to the cultures after three hours, and the cultures were then cultivated for another three hours.

E. coli cultures were pelleted by centrifugation and pellets were frozen at −20° C. Lysates were prepared by freeze-thawing the E. coli pellet in PBS 0.05% Tween 20 with Complete EDTA-free protease inhibitor (Roche, Basel, Switzerland), 25 U/ml Benzonase (Sigma-Aldrich, St Louis, Mo.) and 1 KU/ml rLysozyme (EMD Chemicals, Darmstadt, Germany) and incubation for 10 min at room temperature with shaking

### Site-Directed Mutagenesis

Site-directed mutagenesis was performed using the QuikChange II mutagenesis kit (Stratagene) according to the manufacturer's recommendations with primers carrying the specific mutation. The new CHIPS variants were sequence verified and transformed into E. coli BL-21 Star(DE3)pLysS (Invitrogen) for protein expression.

### Affinity Purification of Human Anti-CHIPS₃₁₋₁₁₃ IgG

Purified CHIPS₃₁₋₁₁₃ was coupled to CNBr activated Sepharose 4B (Amersham Biosciences, Uppsala, Sweden) and packed on a Tricon 5/20 column (Amersham Biosciences) according to manufacturer's instructions. Affinity purification was performed on an ÄKTA Prime system (Amersham Biosciences) according to the manufacturer's protocol. Total human IgG (1 g) (IV-IgG) (Sanquin, Amsterdam, The Netherlands) was run over the column. Bound human IgG was eluted with 0.1 M glycine pH 3.0 and the pH neutralized with 1 M Tris, pH 8.0. Eluted fractions containing protein were pooled and buffer was changed to PBS on PD-10 columns (Amersham Biosciences).

### Phage Selection

Random mutagenesis libraries and FIND® libraries were cloned into the SfiI and NotI sites of the phagemid pFAB75 (Johansen et al., 1995) and transformed into E. coli TOP10 F′(Invitrogen) for expression on phage particles. Phage stocks were prepared according to standard protocols, using VSCM13 (Stratagene) as helper phage (Cicortas Gunnarsson et al., 2004). Positive selections were performed on a biotinylated C5aR peptide consisting of amino acids 7-28 (biotin-05aR peptide) (AnaSpec, San José, Calif.) at a final concentration of 10⁻⁷ M and streptavidin-coated magnetic Dynabeads (Invitrogen). The mixture was incubated for 1 hour on rotation at room temperature, followed by extensive washing in PBS 0.05% Tween 20 with 1% bovine serum albumin (BSA) (selection buffer). Elution of peptide binders was performed with 1M Glycine 0.1% BSA, pH 2.2, followed by addition of 1M Tris pH 9.0 to neutralize the eluate. The selection protocol was then repeated once as described above. Directly after the second round of positive selection, CHIPS phage stocks were subjected to a round of negative selection for human anti-CHIPS₃₁₋₁₁₃ IgG binding. Estapor 0.83 μm magnetic beads (Bangs-Laboratories Inc., Fishers, Ind.) coated with human anti-CHIPS₃₁₋₁₁₃ IgG were washed three times in selection buffer and then blocked in selection buffer for 1 hour on rotation at room temperature. The eluate from the positive selection was added to the beads and they were incubated for another 15 minutes at room temperature. After separation on a magnet, the supernatant was saved and used for infection of exponentially growing E. coli TOP10 F′ and phagemids were purified from the E. coli.

### ELISA

ELISA was used for screening and characterization of binding throughout the study. Maxisorb clear or white 96 or 384 well plates (Nunc, Roskilde, Denmark) were coated overnight at 4° C. with the specific protein or antibody in PBS. Incubations were carried out in a volume of 100 or 25 μl for 1 hour at room temperature if not described differently, always followed by washing three times with PBS 0.05% Tween 20. Super Signal ELISA Pico Chemiluminescent Substrate (Pierce, Rockford, Ill.) was used and luminescence was measured.

### Analysis of Protein Expression

For quantification of expressed proteins, plates were coated with 3 μg/ml monoclonal anti-CHIPS antibody 2H7 recognizing a peptide of CHIPS amino acids 24-30 (Haas et al., 2004). Plates were blocked in PBS 0.05% Tween 20 with 3% milk powder, washed and incubated with dilutions of lysates from the ΔC CHIPS variants. Binding was detected with 3 μg/ml polyclonal rabbit anti-CHIPS N-terminal IgG (IgG produced by immunization of a rabbit with a KLH-coupled synthetic peptide corresponding to CHIPS N-terminal amino acids 1-14) and horseradish peroxidase (HRP) conjugated goat anti-rabbit IgG (Southern Biotech, Birmingham, Ala.).

### Analysis of Anti-CHIPS IgG Binding

For detection of binding of human anti-CHIPS₃₁₋₁₁₃ IgG to CHIPS ΔC variants, plates were coated, blocked and incubated with E. coli lysates as described for analysis of protein expression. Affinity purified human anti-CHIPS₃₁₋₁₁₃ IgG was added and binding was detected with goat-anti-human IgG HRP (Jackson ImmunoResearch, West Grove, Pa.).

During initial screenings, single point measurements were performed on the CHIPS lysates and were compared to a titration curve of Wt CHIPS₁₋₁₂₁. Results were correlated to the results from the expression ELISA. Full titration curves were made for a limited number of variants in later screenings/characterizations.

### Analysis of Peptide Binding

In order to measure the binding of CHIPS variants towards the C5aR peptide, 5 μg/ml Streptavidin (Sigma-Aldrich) was coated. Furthermore, the biotin-05aR peptide (Anaspec) was added to a final concentration of 0.3 μg/ml after washing and blocking the plates (2% BSA in PBS 0.05% Tween 20). Plates were then incubated with CHIPS lysates and detection was performed with 1 μg/ml mAb 2H7 and HRP-conjugated rabbit anti-mouse IgG (Dako, Glostrup, Denmark).

Analysis of Anti-CHIPS IgG Binding in Competition with CHIPS₁₋₁₂₁

Five-fold dilution series of the CHIPS variants were preincubated with 60 ng/ml affinity purified human anti-CHIPS₃₁₋₁₁₃ polyclonal IgG in a polypropylene plate (Nunc) for 2 hours at room temperature. Purified wt CHIPS₁₋₁₂₁ was coated in the ELISA plate. After blocking with 4% BSA in PBS-0.05% Tween-20, the antibody/CHIPS variant mixtures were added to the plate and further incubated for 2 hours at room temperature. Detection was performed with goat-anti-human IgG HRP and o-phenylenediamine dihydrochloride (OPD) substrate.

### Analysis of Serum IgG Binding

IgG from human pooled serum was tested for reactivity with CHIPS variants in ELISA. The plate was coated with equimolar amounts of the proteins or PBS. After blocking in PBS-0.05% Tween-20 with 3% milk powder, serially diluted human serum was added. IgG binding to CHIPS variants was detected with rabbit anti-human IgG-HRP (Dako).

### Biological Assays

### Binding to the Human C5aR

Human neutrophils were prepared from buffy coats obtained from Lund University Hospital (Lund, Sweden), by Percoll (Sigma-Aldrich) density gradient centrifugation. Remaining erythrocytes were lysed with ice-cold H₂O for 30 s. Cells were finally collected in RPMI 1640 (Lonza, Basel, Switzerland) with 0.05% BSA.

Binding to the human C5aR was studied on human neutrophils as well as on the stably transfected cell line U937/C5aR, a generous gift from Dr. E. Prossnitz (University of New Mexico, Albuquerque, N. Mex.). Cells were grown in 75 cm² cell culture flasks in a 5% CO₂ incubator at 37° C. and were maintained in RPMI 1640 medium with L-glutamine (Lonza) and 10% fetal bovine serum (FBS) (Lonza). Binding to the C5aR was analyzed in two ways by flow cytometry. In the first method, dilution series of ΔC CHIPS variants (expressed by the Expressway Cell-Free E. coli Expression System from Invitrogen) were incubated with cells and CHIPS binding was detected by the 2H7 monoclonal anti-CHIPS antibody, followed by a R-phycoerythrin (RPE) labeled goat anti-mouse immunoglobulin (Dako). In the second method, CHIPS ΔC variants were incubated with cells as above, then the degree of inhibition of binding was quantified by adding a monoclonal anti-05aR antibody and the RPE-labeled goat anti-mouse immunoglobulin to the cells.

### C5aR Inhibition and Inhibition of Neutrophil Migration

C5a induced calcium mobilization in human neutrophils was studied by flow cytometry. 5×10⁶/ml neutrophils were incubated with 2 μM Fluo-3AM (Sigma-Aldrich) in RPMI 1640 medium with 0.05% BSA for 30 min at room temperature (RT), followed by washing and resuspension in RPMI 1640 with 0.05% BSA. Cells were then preincubated with a 3-fold dilution series of purified CHIPS variants (re-cloned into the ΔN/C format) at room temperature for 30 min and C5a (Sigma-Aldrich) (final concentration 3 nM) was added to induce calcium release. This was measured by means of fluorescence on a FACScalibur flow cytometer (BD Biosciences, San José, Calif.).

C5a induced migration of human neutrophils (chemotaxis) was measured in a transwell system (Neuro Probe, Gaithersburg, Md.). Therefore 5×10⁶/ml human neutrophils were labelled with 4 μM Calcein-AM (Sigma-Aldrich), washed in Hank's balanced salt solution (HBSS) with 1% human serum albumin (HSA) and resuspended in HBSS with 1% HSA. Cells were further incubated for 15 minutes at RT with a titration of purified CHIPS ΔN/C variants. C5a was added to the lower compartment of the wells to a final concentration of 1 nM. Labelled cells were added to the upper compartments. Plates were incubated for 30 minutes at 37° C. with 5% CO₂. Then filters were rinsed with PBS to remove non-migrating cells and fluorescence was measured at an excitation of 485 nm and emission of 530 nm in a fluorescence plate reader.

### Thermal Denaturation by Circular Dichroism (CD) Spectroscopy

The CD signal at 212 nm was monitored during thermal unfolding of the CHIPS variants from 4-85° C. at a scan rate of 1° C./min, response of 16 s and bandwidth of 1 nm. The protein concentration was 0.5 mg/ml in PBS pH 7.2 and a quartz cuvette with 1 mm pathlength was used. To investigate the reversibility, a thermal scan from 85-4° C. was monitored after the upward scan. Structural changes were determined from far-UV CD spectra, at 4 or 85° C., before and after each thermal scan. Spectra were recorded between 250-195 nm, the scan rate was 20 nm/min, the response 8 s and the bandwidth 1 nm. All CD spectroscopy was carried out on a Jasco (Jasco Inc., Easton, Md.) J-720 spectropolarimeter with a JASCO PTC-343 Peltier type thermostated cell holder. Since the thermal unfolding was irreversible for all variants no thermodynamic stability could be obtained. However, since unfolding was monitored at the same speed for all variants the Tₘ gives comparative thermal stabilities between the variants. The Tₘ was obtained by fitting eq. 1 to CD data.

$${ɛ}_{obs} = \frac{\begin{matrix}( {k}_{N} \cdot T + {b}_{N} ) + \\ ( {k}_{U} \cdot T + {b}_{U} ) \cdot {e}^{- ( A ( 1 - ( T / {T}_{m} ) ) + 3000 ( T - {T}_{m} - T \cdot ln ( T / {T}_{m} ) ) ) / RT}\end{matrix}}{( 1 + {e}^{- ( A ( 1 - ( T / {T}_{m} ) ) + 3000 ( T - {T}_{m} - T \cdot ln ( T / {T}_{m} ) ) ) / RT} )} \qquad ( eq . 1 )$$

In eq. 1, ε_{obs} is the observed ellipticity at 212 nm, k_{N}, b_{N}, k_{U} and b_{U} define the baselines of the native and unfolded states respectively. A is a parameter in the fitting process but has no value for an irreversible unfolding, T is the temperature in Kelvin and R is the gas constant. In the equation, the protein is assumed to follow a two-state denaturation process and have a constant ΔC°ₚ in the temperature region so that the denaturation follows Gibbs-Helmholtz equation. For such an unfolding the parameter A is ΔH° and 3000 is an estimated measure for ΔC°ₚ but these parameters have no relevance for an irreversible unfolding.

### Molecular Modelling

Modelling was performed by the use of the available CHIPS₃₁₋₁₂₁ NMR structure (PDB code: 1×EE) (Haas et al., 2005) and the PyMol molecular graphics program (DeLano, 2008).

### Results

Strategy to Create CHIPS Variants with Low Antibody Binding

The evolution for decreased anti-CHIPS antibody interaction, yet preserved C5aR blocking activity was performed in one round of random mutagenesis and three rounds of FIND® recombination, followed by computational analysis and rational design (FIG. 4). A shorter CHIPS variant, truncated at both the N- and C-terminus (CHIPS ΔN/C) and single mutants K61A, K69A and K100A that had previously shown to be less prone to bind human anti-CHIPS antibodies (data not shown) were chosen as starting material for the optimization process. The first 30 N-terminal amino acids in CHIPS were kept as a recognition sequence (that was not subjected to mutagenesis or recombinations) for capture antibodies in ELISA. Selected clones were then re-cloned into the truncated (CHIPS ΔN/C) format before characterization of biological activity (C5aR inhibition).

In order to increase the probability to find new CHIPS variants with decreased antibody binding, several ELISAs were applied for studying the interaction between CHIPS and affinity purified anti-CHIPS₃₁₋₁₁₃ IgG. Several screening rounds were performed in ELISA for each of the libraries 1, 3 and 4. The primary screening of each round was performed by one-point measurements, whereas the assays were more comprehensively performed in later rounds of screening by making full titration curves for each of the selected mutants.

Furthermore, to preserve the biological functionality of the new CHIPS variants during selections and screening, binding to a peptide of the C5aR N-terminal amino acids 7-28 was continuously monitored. Residues 10-18 of the C5aR have previously been shown to be the binding domain for CHIPS (Postma et al., 2005). Tyrosine residues 11 and 14 of the C5aR have been shown to be sulphated, which was shown to be critical for C5a dependent activation of the C5aR (Farzan et al., 2001). A recent study on CHIPS binding to peptides of the C5aR N-terminus, stresses the role for sulphated tyrosines in positions 11 and 14 for CHIPS binding and shows that CHIPS binds with high affinity to sulphated peptides of the C5aR N-terminus (Ippel et al., 2009).

### Random Mutagenesis Libraries and Screening

Diversity was introduced into the CHIPS ΔC sequence by random mutagenesis. Four libraries with different mutation frequencies were created (details of the libraries are described in Table 1). All four libraries were subjected to phage selection; first for C5aR peptide binding (positive selection), followed by selection for decreased anti-CHIPS antibody binding (negative selection). Supernatants from the negative selection were pooled, allowed to infect E. coli and phagemids were purified. The CHIPS encoding sequences from the pool of mutants was re-cloned into the expression vector pRSET B and 360 CHIPS variants were subsequently expressed in plate format and screened in ELISA for decreased anti-CHIPS₃₁₋₁₁₃ IgG binding (FIG. 5 A). The 64 clones with lowest anti-CHIPS₃₁₋₁₁₃ IgG binding were further analyzed for retained C5aR peptide binding in ELISA. From these, 30 clones with high C5aR peptide binding were selected for further analysis of decreased anti-CHIPS₃₁₋₁₁₃ IgG binding by making full titration curves in ELISA. Finally, 9 clones with significantly reduced anti-CHIPS₃₁₋₁₁₃ IgG binding, yet preserved C5aR peptide binding were selected for DNA recombination by FIND®.

### FIND® Libraries and Screening

### FIND® Round 1

Two libraries with different recombination frequencies (i.e. different numbers of cross overs) were created from the 9 clones (containing in total 18 amino acid substitutions) selected in the random mutagenesis step (Table 1). Library 2.2 was created by FIND® using error-prone conditions to increase the diversity in the parent polynucleotides used in the library.

The libraries were subjected to phage selection as described above and supernatants from the negative selection were pooled, and phagemids were purified from E. coli. This pool of DNA was used as starting material for the second round of FIND®.

### FIND® Round 2

In the second round of FIND® recombinations, one library was created (Table 1). 6.3×10³ clones were expressed in plate format and screened. The 320 clones that showed maximum 70% of wt CHIPS₁₋₁₂₁ binding to the anti-CHIPS₃₁₋₁₁₃ IgG and at least 80% of wt CHIPS₁₋₁₂₁ binding to the C5aR peptide in ELISA were selected for a second round of screening for lower anti-CHIPS₃₁₋₁₁₃ IgG binding in ELISA (FIG. 5 A).

Of these, 40 clones showed <40% binding compared to wt CHIPS₁₋₁₂₁ and were then further analyzed in a dose-dependent set up in ELISA. The EC₅₀ value (i.e. the concentration of each CHIPS variant mediating half-maximal binding) and plateau value of each variant were determined and compared to the values of wt CHIPS₁₋₁₂₁. The 12 clones that showed at least 2.4 higher EC₅₀ and maximum 54% of wt CHIPS₁₋₁₂₁ plateau value in anti-CHIPS₃₁₋₁₁₃ IgG binding, were chosen for a last round of FIND® recombination.

### FIND® Round 3

Two libraries were created in the final round of FIND® recombinations. The first library was based on six of the selected clones with 14 amino acid changes represented and the second library was made from all 12 clones selected during the previous round of FIND® (in total 25 amino acid changes) (Table 1). Both libraries were designed by the use of two repeated rounds of FIND® without any selection or screening in between, which generated a higher frequency of recombined clones (92%) than in the previous libraries (Table 1). 9.6×10³ clones were expressed in plate format and screened in ELISA for decreased human anti-CHIPS₃₁₋₁₁₃ IgG binding. 1000 clones showed maximum 10% of wt CHIPS₁₋₁₂₁ binding to anti-CHIPS₃₁₋₁₁₃ IgG and were further analyzed for C5aR peptide binding in ELISA. The 96 clones that showed at least 95% binding to the C5aR peptide as compared to wt CHIPS₁₋₁₂₁ (FIG. 5 A, C), were selected for further analysis in ELISA for decreased anti-CHIPS₃₁₋₁₁₃ IgG binding and in flow cytometry for retained C5aR binding. The clones showed on average 7.1% binding of anti-CHIPS₃₁₋₁₁₃ IgG as compared to wt CHIPS₁₋₁₂₁. The most improved clone showed 2.5% binding as compared to wt CHIPS₁₋₁₂₁ (FIG. 5 B). The distribution of the 96 clones is shown in FIG. 5 C.

After sequencing, 42 unique clones were identified. All 42 clones showed <10% binding to the anti-CHIPS₃₁₋₁₁₃ IgG as compared to wt CHIPS₁₋₁₂₁ after the last round of screening. These clones were expressed by a cell-free protein expression system to improve the yield of protein and the products were characterized further by ELISA and flow cytometry. The 10 clones showing the highest binding to the human C5aR as well as low binding to anti-CHIPS₃₁₋₁₁₃ IgG (<13% of CHIPS ΔC) (FIG. 6), were selected for further mutagenesis. During this thorough characterization, a small number of clones showed higher binding towards the anti-CHIPS₃₁₋₁₁₃ IgG than measured during the previous screening, hence the cut-off value was set to 13% instead of the previous 10%.

### Molecular Modelling and Rational Design

To decrease the interaction with anti-CHIPS₃₁₋₁₁₃ IgG even further, 27 new CHIPS variants were created by site-directed mutagenesis of four of the 10 selected clones (table 3 and 4). By introducing new mutations into these specific clones, all 12 mutated positions identified during directed evolution were represented. The site-directed mutagenesis was designed by analyzing the protein structure, with special attention paid to the structural role of the mutated amino acid residues generated during the directed evolution. New substitutions were suggested in some of these mutated positions and these were also joined in new combinations compared to the clones generated during directed evolution.

Mutants were analyzed for anti-CHIPS₃₁₋₁₁₃ IgG binding in ELISA and for C5aR binding and blocking (inhibition of Ca²⁺ release upon C5a dependent activation of the C5aR) by flow cytometry. 16 clones with a C5aR blocking IC₅₀ value of maximum four times that of wt CHIPS₁₋₁₂₁were selected for further characterizations. Clones with a glycine or alanine in position 112 showed higher binding to the C5aR than clones with a valine in this position. For this reason, V112 was mutated to an alanine in the final clones.

### Characterization of CHIPS Variants

In order to study the interaction between the selected CHIPS variants and anti-CHIPS₃₁₋₁₁₃ IgG as well as the human C5aR, the final 16 clones were re-cloned into the CHIPS ΔN/C format and the proteins were produced and purified from inclusion bodies through extensive washing of the inclusion bodies, solubilisation, refolding (dropwise addition of the protein solution into PBS) and gel filtration as described previously (Gustafsson et al., 2009). After further analyses of CHIPS binding to serum IgG by ELISA and biological functionality by flow cytometry, 7 clones could finally be selected as the most promising candidates. This choice was based on the following criteria: Serum IgG titer at a maximum of 2.5% and C5aR blocking activity of at least 25%, of that observed for wt CHIPS₁₋₁₂₁. These clones were further characterized by studying inhibition of neutrophil migration (chemotaxis) and by determining Tₘ values by CD (Table 2). The temperature denaturations show that all clones have a high melting temperature compared to CHIPS ΔN/C. Some variants show a minor transition at a low temperature and a major transition at a high temperature, indicating partial unfolding at the low temperature. CHIPS ΔN/C shows a reversible unfolding while all seven clones show an irreversible thermal unfolding. This suggests a higher aggregation propensity of the clones in the unfolded state compared to CHIPS ΔN/C.

FIG. 7 shows a sequence alignment of the top seven clones obtained after random mutagenesis, FIND® and rational design. The clones contain between five and eight mutations per sequence. Three mutated positions are located in the α-helix, one in the loop between the β₁ and β₂ strands, two in the loop between the β₂ and β₃ strands, one in β strand 3, one in the loop between the β₃ and β₄ strands and two in β strand 4. More specifically, positions K40, D42, N77, K100, N111 and G112 are mutated in four or more clones in different combinations with mutations in positions K50, K69, K92 and K105. Substitutions N77Y, N111K and G112A are the most common among the clones, represented in six out of seven clones. The specific combination of the mutations in the top seven clones was found to be favourable in terms of low IgG titer, high C5aR blocking activity as well as low IC₅₀ in inhibition of chemotaxis.

### DISCUSSION

Previously, medicine has relied primarily on chemically or synthetically produced small molecule drugs. Recently, protein drugs have become more important along with the development of recombinant DNA technology, high throughput screening and proteomics. Although a certain characteristic of a protein can be of interest for drug development, other properties might need to be improved in order to design a promising drug candidate. Today, there are a number of drugs (approved or in clinical phase trials) that have been optimized by the use of protein engineering. Tissue plasminogen activator (t-PA) has been improved several times to finally have a longer half-life in serum as well as higher specificity for fibrin (Keyt et al., 1994). This engineered version of tPA (TNKase®) is now approved for the treatment of acute myocardial infarction. ANYARA is a superantigen coupled antibody with tumor specificity, currently in clinical trials. The antigenicity of the superantigen SEA (Staphylococcal enterotoxin A) has been decreased to make ANYARA a more attractive anti-tumor drug candidate (Erlandsson et al., 2003).

In combination with a well designed screening method, directed evolution can be utilized to improve almost any characteristic of a protein, i.e. improved affinity, higher potency or decreased immunogenicity. However, when improving a specific property of interest, it is important to continuously monitor other significant characteristics of the protein that might also be altered during the optimization of the specific property.

In this study, we were able to decrease the interaction with human antibodies to only 0.5% of wt CHIPS₁₋₁₂₁ while retaining C5aR blocking activity. This was achieved by continuously monitoring the C5aR binding during the rounds of directed evolution and screening to ensure that this property was not lost during the optimization process. Moreover, to increase the probability to find new CHIPS variants with decreased antibody binding, several methods for verifying this property were applied during the rounds of screening.

Directed evolution (random mutagenesis and FIND®) was applied in combination with computational/rational design to improve the CHIPS molecule towards lower interaction with specific human antibodies. Diversity was first introduced into the sequence by random mutagenesis, followed by three rounds of FIND® recombinations performed sequentially. Without need for prior knowledge of the antigenic epitopes in CHIPS, the mutations found to be beneficial in the previous round were recombined to form new CHIPS variants and antibody binding was shown to decrease with every round. After the last round of FIND®, the best clones displayed a binding of human anti-CHIPS₃₁₋₁₁₃ IgG that was reduced to only 2.5% of the binding towards wt CHIPS₁₋₁₂₁. This was a significant decrease in binding achieved by the application of directed evolution. However, to decrease the binding even further, site-directed mutagenesis was designed by molecular modelling and additional mutations were introduced. This was accomplished by analyzing the structural distribution of the positions found to be of importance in the directed evolution process.

The combination of mutations in the top seven clones is responsible for the unique properties of these variants. In an attempt to investigate the contribution of the different mutated residues, a structural analysis of the most frequently mutated positions among the final seven clones; D42, N77, N111 and G112 was applied. D42 is an amino acid in the α-helix that seems to be important for intramolecular interactions. Substitution to a valine (V) potentially breaks the H—H bond formed between D42 and R46. This change may alter the structure of the CHIPS molecule and possibly also change an antibody epitope. The introduction of the hydrophobic valine at position 42 seems to increase the stability of the molecule. Most likely, this hydrophobic residue fits well into the interior of the structure and stabilizes the hydrophobic core and that may be the reason why it is represented in six out of the seven selected clones. However, the mutation might affect the reversibility and the aggregation propensity in the unfolded state due to the increased hydrophobicity. N77, is mutated to a tyrosine (Y) in six of the clones and to a histidine (H) in one clone. It is exposed in the β2-β3 loop and could be directly involved in antibody binding. When comparing N77Y and N77H it appears that the tyrosine increases the stability compared to the histidine in this position. On the other hand clone 376, with a histidine in this position, has a better preserved biological function (inhibition of chemotaxis) as compared to clone 335 that is identical apart from a tyrosine in position 77. N111 is an exposed residue in β4. This position becomes more positively charged upon substitution to lysine (K), this is a significant change of the surface that was shown to be beneficial in six out of the seven clones. G112 in β4 is not particularly exposed. A small amino acid was found to be advantageous in this position. If a large amino acid, such as valine, is inserted in this position, it might collide with M93, and as a result the structure may be affected. Changing the G112V mutation, selected during directed evolution, to an alanine (A) was found to be beneficial for preserving C5aR blocking activity in all clones carrying the G112V mutation. Interestingly, three of the seven top clones (variants 335, 338 and 377) were the same as clones found during directed evolution, but with a substitution to A in position 112 instead of V.

The approach to combine random mutagenesis or directed evolution with computational/rational design has also been successfully applied by others (Buskirk et al., 2004). For example, mutagenesis can first been utilized to provide information on residues important to mutate. This way, mutagenesis can be directed from a randomized point of view instead of being based on rational choices (Lingen et al., 2002).

The removal of antibody epitopes is relevant in several disciplines within immunology. In allergy research, IgE epitopes are removed to create hypoallergenic allergen derivatives to be used as candidate vaccines (Linhart et al., 2008; Mothes-Luksch et al., 2008; Szalai et al., 2008; Vrtala et al., 2004). To our knowledge, this work has been performed by epitope mapping and subsequent genetic engineering or by the design of mosaic proteins or hybrid molecules to achieve derivatives with reduced allergenic activity and preserved immunogenicity. Our results demonstrate that epitopes for human IgG can be efficiently reduced in a protein by the use of directed evolution and computational/rational design. This random approach to eliminate antibody epitopes might be useful to apply in e.g. the design of hypoallergenic allergen derivatives.

In conclusion, by the use of directed evolution, computational analysis and rational design we have generated new CHIPS molecules with decreased interaction with specific human IgG without affecting the interaction between CHIPS and the C5aR to a high extent. This work has resulted in CHIPS variants that are better suited to therapeutic use than the wt CHIPS₁₋₁₂₁ protein, because they will most likely not form complexes with human antibodies, and thereby they will be better tolerated and function more efficient as C5aR antagonists in human serum. Out of these, one variant (376) was identified having unexpectedly advantageous properties. This clone was designated ADC-1004.

### REFERENCES

Buskirk, A. R., Landrigan, A. and Liu, D. R. (2004) Chem Biol, 11, 1157-1163.
Cicortas Gunnarsson, L., Nordberg Karlsson, E., Albrekt, A. S., Andersson, M., Holst, O. and Ohlin, M. (2004) Protein Eng Des Sel, 17, 213-221.
Dahlen, E., et al. (2008) J Immunotoxicol, 5, 189-199.
De Haas, C. J., Veldkamp, K. E., Peschel, A., Weerkamp, F., Van Wamel, W. J., Heezius, E. C., Poppelier, M. J., Van Kessel, K. P. and Van Strijp, J. A. (2004) J Exp Med, 199, 687-695.
DeLano, W. L. (2008) The PyMOL Molecular Graphics System. Delano Scientific LLC, Palo Alto, Calif.
Erlandsson, E., et al. (2003) J Mol Biol, 333, 893-905.
Farzan, M., Schnitzler, C. E., Vasilieva, N., Leung, D., Kuhn, J., Gerard, C., Gerard, N. P. and Choe, H. (2001) J Exp Med, 193, 1059-1066.
Gafvelin, G., Parmley, S., Neimert-Andersson, T., Blank, U., Eriksson, T. L., van Hage, M. and Punnonen, J. (2007) J Biol Chem, 282, 3778-3787.
Gustafsson, E., Forsberg, C., Haraldsson, K., Lindman, S., Ljung, L. and Furebring, C. (2009) Protein Expr Purif, 63, 95-101.
Haas, P. J., de Haas, C. J., Kleibeuker, W., Poppelier, M. J., van Kessel, K. P., Kruijtzer, J. A., Liskamp, R. M. and van Strijp, J. A. (2004) J Immunol, 173, 5704-5711.
Haas, P. J., et al. (2005) J Mol Biol, 353, 859-872.
Heller, T., et al. (1999) J Immunol, 163, 985-994.
Ippel, J. H., de Haas, C. J., Bunschoten, A., van Strijp, J. A., Kruijtzer, J. A., Liskamp, R. M. and Kemmink, J. (2009) J Biol. Chem.
Johannes, T. W. and Zhao, H. (2006) Curr Opin Microbiol, 9, 261-267.
Johansen, L. K., Albrechtsen, B., Andersen, H. W. and Engberg, J. (1995) Protein Eng, 8, 1063-1067.
Keyt, B. A., et al. (1994) Proc Natl Acad Sci USA, 91, 3670-3674.
Knecht, W., et al. (2006) Febs J, 273, 778-792.
Leung, D. W., Chen, E. and Goeddel, D. V. (1989) Technique, 11-15.
Lingen, B., Grotzinger, J., Kolter, D., Kula, M. R. and Pohl, M. (2002) Protein Eng, 15, 585-593.
Linhart, B., Mothes-Luksch, N., Vrtala, S., Kneidinger, M., Valent, P. and Valenta, R. (2008) Biol Chem, 389, 925-933.
Mothes-Luksch, N., et al. (2008) J Immunol, 181, 4864-4873.
Postma, B., Kleibeuker, W., Poppelier, M. J., Boonstra, M., van Kessel, K. P., van Strijp, J. A. and de Haas, C. J. (2005) J Biol. Chem.
Postma, B., Poppelier, M. J., van Galen, J. C., Prossnitz, E. R., van Strijp, J. A., de Haas, C. J. and van Kessel, K. P. (2004) J Immunol, 172, 6994-7001.
Reetz, M. T. (2004) Proc Natl Acad Sci USA, 101, 5716-5722.
Rittirsch, D., et al. (2008) Nat Med, 14, 551-557.
Sneeden, J. and Loeb, L. (2003) Random oligonucleotide mutagenesis. In Arnold, F. and Georgiou, G. (eds.), Directed evolution library creation. Methods and protocols. Humana Press, Totowa, N.J., Vol. 231, pp. 65-73.
Stemmer, W. P. (1994a)Proc Natl Acad Sci USA, 91, 10747-10751.
Stemmer, W. P. (1994b)Nature, 370, 389-391.
Szalai, K., et al. (2008) Mol Immunol, 45, 1308-1317.
Wong, T. S., Roccatano, D. and Schwaneberg, U. (2007) Environ Microbiol, 9, 2645-2659.
Wright, A. J., Higginbottom, A., Philippe, D., Upadhyay, A., Bagby, S., Read, R. C., Monk, P. N. and Partridge, L. J. (2007) Mol Immunol, 44, 2507-2517.
Vrtala, S., Focke-Tejkl, M., Swoboda, I., Kraft, D. and Valenta, R. (2004) Methods, 32, 313-320.
Yuan, L., Kurek, I., English, J. and Keenan, R. (2005) Microbiol Mol Biol Rev, 69, 373-392.
Zhao, H. (2007) Biotechnol Bioeng, 98, 313-317.
Zhao, H., Giver, L., Shao, Z., Affholter, J. A. and Arnold, F. H. (1998) Nat Biotechnol, 16, 258-261.

TABLE 1
Library characteristics

| | | Mutations | New | Frequency | Number of | Frequency | Library |
|---|---|---|---|---|---|---|---|
| | | in starting | mutations/ | recombined | recombinations/ | unique | size |
| Round | Library¹ | material² | clone² | sequences (%) | recombined clone | sequences (%) | (clones) |
| 1 (Random | 1.1 | (3) | 6.3 (3.6) | N/A⁴ | N/A⁴ | N/D³ | 2 × 10⁶ |
| mutagenesis) | 1.2 | (3) | 2.2 (1.5) | N/A⁴ | N/A⁴ | N/D³ | 2 × 10⁶ |
| | 1.3 | (3) | 3.1 (2.5) | N/A⁴ | N/A⁴ | 90 | 2 × 10⁶ |
| | 1.4 | (3) | 1.4 (1.1) | N/A⁴ | N/A⁴ | 100 | 2 × 10⁶ |
| 2 (FIND ® round 1) | 2.1 | 26 (18) | 0.2 | 28 | 1.4 | 81 | 2 × 10⁵ |
| | 2.2 | 26 (18) | 2.4 | 48 | 1.6 | 97 | 7 × 10⁵ |
| 3 (FIND ® round 2) | 3.1 | N/D³ | N/D³ | 58 | 1.6 | 88 | 5 × 10³ |
| 4 (FIND ® round 3) | 4.1 | 19 (14) | 0 | 92 | 1.7 | 96 | 5 × 10³ |
| | 4.2 | 36 (25) | 0.04 | 92 | 1.8 | 96 | 1 × 10⁴ |

¹Sequence analysis was performed on 24 clones from each library.
²Mutations shown as base changes with amino acid changes in brackets.
³N/D Not determined
⁴N/A Not applicable

TABLE 2
Characteristics of the final seven clones in comparison to the truncated wt CHIPS₃₁₋₁₁₃

| | | | 95% confidence | | | | |
|---|---|---|---|---|---|---|---|
| | Inhibition | Chemotaxis | interval | | Theoretical | | Comments on |
| | of Ca²⁺ | IC₅₀ | chemotaxis | IgG | isoelectric | | temperature |
| Clone | release | (μg/ml) | IC₅₀ (nM) | titer | point (pl) | Tₘ (° C.) | denaturation |
| Wt CHIPS₁₋₁₂₁ | ++++ | 0.1 | 6.7-10 | 33667 | 9.36 | N/D¹ | N/D¹ |
| CHIPS ΔN/C | +++ | 0.5 | 38-68 | 1826 | 9.70 | 60.1 ± 0.4 | one transition, |
| | | | | | | | reversible |
| 332 | ++(+) | 7.6 | 568-1120 | 694 | 9.67 | 75.7 ± 1.8 | one transition, |
| | | | | | | | irreversible |
| 335 | ++(+) | 2.0 | 118-355 | 611 | 9.74 | 83.9 ± 0.6 | one transition, |
| | | | | | | | irreversible |
| 336 | ++(+) | 8.0 | 297-2370 | 382 | 9.85 | 81.2 ± 0.6 | one transition, |
| | | | | | | | irreversible |
| 338 | +(+) | 2.5 | 233-313 | 179 | 9.67 | 28.2 ± 1.8; | two transitions, |
| | | | | | | 87.0 ± 1.3 | irreversible |
| 376 | ++(+) | 0.5 | 39-60 | 196 | 9.78 | 64.4 ± 0.2 | one transition, |
| | | | | | | | irreversible |
| 377 | ++ | 2.3 | 158-377 | 210 | 9.71 | 30.0 ± 2.8; | two transitions, |
| | | | | | | 85.7 ± 0.8 | irreversible |
| 441 | ++(+) | 5.0 | 316-885 | 784 | 9.67 | 30.2 ± 3.5; | two transitions, |
| | | | | | | 60.2 ± 0.1 | irreversible |

¹N/D Not determined

TABLE 3
Mutations in 10 selected clones after the fourth round of diversification and screening

| Clone | K40 | D42 | K50 | K69 | N77 | D83 | L90 | K92 | K100 | K105 | N111 | G112 |
|---|---|---|---|---|---|---|---|---|---|---|---|---|
| F3.03 | • | • | N | R | Y | • | • | R | • | • | K | V |
| F3.08 | E | V | • | • | Y | • | • | • | R | R | K | V |
| F3.14 | • | • | N | • | Y | • | • | R | • | • | K | V |
| F3.39 | E | V | • | • | Y | • | • | • | • | • | K | V |
| F3.46 | E | V | • | • | Y | • | • | R | • | • | K | V |
| F3.50 | • | • | N | • | Y | • | • | • | • | • | K | V |
| F3.57 | E | V | N | • | Y | • | • | R | • | • | K | V |
| F3.70 | N | • | N | • | Y | • | • | R | • | • | I | • |
| F3.71 | N | • | • | • | Y | G | P | • | • | • | K | V |
| F3.85 | • | • | N | • | Y | • | • | R | R | • | I | • |

TABLE 4
Site-directed mutations made in four of the clones selected after FIND ® recombinations

| Clone | K40 | D42 | | | | N77 | | | | K100 | K105 | N111 | G112 |
|---|---|---|---|---|---|---|---|---|---|---|---|---|---|
| F3.08 | E | V | K50 | N68 | K69 | Y | D83 | L90 | K92 | R | R | K | V |
| S3.01 | • | • | E | • | • | • | • | • | • | • | • | • | • |
| S3.02 | • | • | • | • | A | • | • | • | • | • | • | • | • |
| S3.03 | • | • | • | • | T | • | • | • | • | • | • | • | • |
| S3.04 | • | • | • | • | • | H | • | • | • | • | • | • | • |
| S3.05 | • | • | • | • | • | • | H | • | • | • | • | • | • |
| S3.06 | • | • | • | • | • | • | • | E | • | • | • | • | • |
| S3.07 | • | • | • | • | • | • | • | • | E | • | • | • | • |
| S3.08 | • | • | • | • | • | • | • | • | • | A | • | • | • |
| S3.09 | • | • | • | • | • | • | • | • | • | • | • | • | A |
| S3.10 | • | • | E | • | • | • | • | • | R | • | • | • | • |
| S3.11 | • | • | • | • | • | • | • | • | • | • | • | N | • |
| S3.12 | • | • | • | • | • | • | • | • | • | • | • | • | G |
| S3.13 | • | • | • | H | • | • | • | • | • | • | • | • | • |
| F3.71 | N | | | | | Y | G | P | | | | K | V |
| S3.14 | • | • | • | H | • | • | • | • | • | • | • | • | • |
| S3.23 | • | V | • | • | • | • | • | • | • | • | • | • | • |
| S4.01 | • | V | • | • | • | • | • | • | • | • | • | • | A |
| S4.02 | • | V | • | • | • | • | N | • | • | • | • | • | • |
| S4.03 | • | V | • | • | • | • | • | E | • | • | • | • | • |
| S4.04 | • | V | • | • | • | • | N | E | • | • | • | • | • |
| F3.03 | | | N | | R | Y | | | R | | | K | V |
| S3.15 | • | • | • | H | • | • | • | • | • | • | • | • | • |
| S3.16 | • | • | • | • | • | • | • | • | • | A | • | • | • |
| S3.17 | • | • | • | • | • | A | • | • | • | • | • | • | • |
| S3.18 | • | • | • | • | • | A | • | • | • | A | • | • | • |
| S3.20 | E | • | • | • | • | • | • | • | • | • | • | • | • |
| S3.21 | • | V | • | • | • | • | • | • | • | • | • | • | • |
| S3.22 | E | V | • | • | • | • | • | • | • | • | • | • | • |
| F3.70 | N | | N | • | | Y | | | R | | | I | |
| S3.19 | • | • | • | • | • | • | • | • | • | • | • | K | • |

### Example 3

### Directed Evolution of 3C4 scFv

### 3C4 scFv

For the FIND® experiments, the gene of interest was amplified, using vector specific primers (MWG, Ebersberg, Germany). Unless otherwise stated, PCR reactions contained 1 mM of each primer, 200 mM of each dNTP (New England Biolabs, MA, USA), 1× AmpliTaq reaction buffer, 1.25 U AmpliTaq thermostable DNA polymerase (Applied Biosystems, CA, USA) in a total volume of 100 μl. Standard PCR programs consisted of a denaturing step at 94° C. for 2 min, 25 cycles of 94° C. for 1 min, 60° C. for 1 min and 72° C. for 1 min and finally elongation at 72° C. for 7 minutes.

ssDNA representing sense and antisense strands was prepared from 3.10E5, 2.7G5, 2.10H3 or 3.10D1 (4 mutants of the 3C4 scFv). A PCR product was produced using one biotinylated and one unbiotinylated primer, the biotin thus being coupled either to the sense or the antisense strand. The PCR product was immobilized on a column containing streptavidin-conjugated magnetic beads placed in the magnetic field of a μMACS separator (beads and separator both from Miltenyi Biotec, Bergisch Gladbach, Germany). The PCR product was denatured with 0.1M NaOH and the eluted ssDNA was collected. The obtained ssDNA was analyzed by agarose gel (Cambrex, Md., USA) electrophoresis, purified using Recochip (TaKaRa, Shiga, Japan) according to manufacturer's recommendations, and finally ethanol precipitatated.

The FIND® experiments were initiated by fragmenting sense and antisense ssDNA, respectively, with Exonuclease I (Exo I) (100 U/μg DNA, New England Biolabs, MA, USA) (Lehman et al., 1964) for 10 min, Exonuclease V (Exo V) (25 U/μg DNA, USB, OH, USA) (Anai et al., 1970a; Anai et al., 1970b) for 30 min and Exonuclease VII (Exo VII) (5 U/μg DNA, USB, OH, USA) (Chase et al., 1974a; Chase et al., 1974b) for 30 min in separate tubes under conditions recommended by the manufacturer. The exonuclease catalyzed reaction was stopped by heat inactivation at 96° C. for 10 minutes.

The ssDNA fragments resulting from the exonuclease digestions (20 ng from each digestion) were recombined and assembled into full-length product polynucleotides using a first (error-prone) PCR reaction (PCR 1) consisting of a denaturing step at 94° C. for 60 s, 25 cycles of 94° C. for 30 s, 45° C. for 30 s and 72° C. for 60 s and finally an elongation at 72° C. for 7 minutes, without added primers in a 50 μl total volume with 0.2 mM dATP, 0.2 mM dGTP, 1 mM dTTP, 1 mM dCTP, 7 mM MgCl₂, 0.5 mM MnCl₂, and 1.25 U of AmpliTaq DNA.

The material from PCR 1 was then amplified in a second PCR reaction (PCR 2) in two different ways:
1. Conventional (i.e. non-error-prone) PCR 2a reaction consisting of a denaturing step at 94° C. for 60 s, 15 or 20 cycles of 94° C. for 30 s, 55° C. for 30 s and 72° C. for 60 s and finally elongation at 72° C. for 7 minutes, with 0.8 mM of each primer included.
2. Error-prone PCR 2b reaction consisting of a denaturing step at 94° C. for 60 s, 20 cycles of 94° C. for 30 s, 55° C. for 30 s and 72° C. for 60 s and finally elongation at 72° C. for 7 minutes with 0.8 mM of each primers in a 50 μl total volume with 0.2 mM dATP, 0.2 mM dGTP, 1 mM dTTP, 1 mM dCTP, 7 mM MgCl₂, 0.5 mM MnCl₂, and 1.25 U of AmpliTaq DNA.

The FIND EP1 and EP2 libraries were digested with SfiI/NotI and ligated into the SfiI/NotI sites of pFAB75 (Jan Engberg, Department of Pharmacology, Copenhagen, Denmark). Individual clones were sequences and analysed for recombinations and newly introduced mutations.

### Results

Establishment of Mutated Versions of scFv 3C4

3 different libraries of mutants have been generated:
a. FIND1.4EP1 library using the following clones in FIND experiments; 3.10E5, 2.7G5, 2.10H3 and 3.10D1. The first PCR was performed under error-prone conditions (i.e. PCR 1 followed by PCR 2a conditions). 55% of the clones contained recombinations and 0.7 mutations/gene were introduced.
b. FIND1.4EP2 library using the following clones in FIND experiments; 3.10E5, 2.7G5, 2.10H3 and 3.10D1. Both PCR I and II were performed under errorprone conditions (i.e. PCR 1 followed by PCR 2b conditions). All of the sequenced clones contained recombinations and 3.5 mutations/gene were introduced.
c. FIND1.8 library using the following clones in FIND experiments; 3.10E5, 2.7G5, 3.7D9, 3.3D7, 2.10H3, 3.7C10, 3.9G2 and 3.10D1.60% of the clones contained recombinations and 0.8 mutations/gene were introduced using standard FIND conditions (i.e. as described in WO 02/48351, WO 03/097834 and WO 2007/057682).

### CONCLUSIONS

Use of error-prone PCR during the amplification stage resulted in increased mutation levels in the resulting library.

### Example 4

### Directed Evolution of CHIPS

For the FIND® experiments, the gene of interest was amplified, using vector specific primers (MWG, Ebersberg, Germany). Unless otherwise stated, PCR reactions contained 0.5 mM of each primer, 200 mM of each dNTP (New England Biolabs, MA, USA), 1× AmpliTaq reaction buffer, 1.25 U AmpliTaq thermostable DNA polymerase (Applied Biosystems, CA, USA) in a total volume of 50 μl. Standard PCR programs consisted of a denaturing step at 94° C. for 2 min, 35) cycles of 94° C. for 1 min (30 s), 54° C. for 30 s and 72° C. for 30 s and finally elongation at 72° C. for 7 minutes.

ssDNA representing sense and antisense strands was prepared from CHIPS: 3H1, 2C9, 7E4, 6E1, 7B3, 2D5, 4E4, 1F8 and 5H. A PCR product was produced using one biotinylated and one unbiotinylated primer, the biotin thus being coupled either to the sense or the antisense strand. The PCR product was immobilized on a column containing streptavidin-conjugated magnetic beads placed in the magnetic field of a μMACS separator (beads and separator both from Miltenyi Biotec, Bergisch Gladbach, Germany). The PCR product was denatured with 0.1M NaOH and the eluted ssDNA was collected. The obtained ssDNA was analyzed by agarose gel (Cambrex, Md., USA) electrophoresis, purified using Recochip (TaKaRa, Shiga, Japan) according to manufacturer's recommendations, and finally ethanol precipitatated.

The FIND® experiments were initiated by fragmenting sense and antisense ssDNA, respectively, with Exonuclease I (Exo I) (100 U/μg DNA, New England Biolabs, MA, USA) (Lehman et al., 1964) for 10 min, and Exonuclease VII (Exo VII) (10 U/μg DNA, USB, OH, USA) (Chase et al., 1974a; Chase et al., 1974b) for 30 min in separate tubes under conditions recommended by the manufacturer. The exonuclease catalyzed reaction was stopped by heat inactivation at 96° C. for 10 minutes.

The ssDNA fragments resulting from the exonuclease digestions (30 ng from each digestion, fw/rev ExoI/ExoVII; in total 120 ng) were either recombined in a PCR reaction as described above without primers (library 2.1) or in a first PCR reaction (PCR 1) consisting of a denaturing step at 94° C. for 2 min, 25 cycles of 94° C. for 30 s, 50° C. for 45 s and 72° C. for 60 s and finally an elongation at 72° C. for 7 minutes, without added primers in a 50 μl total volume with 0.5 mM dATP, 0.5 mM dGTP, 1 mM dTTP, 1 mM dCTP, 7 mM MgCl₂, 0.5 mM MnCl₂, and 1.25 U of AmpliTaq DNA polymerase (library 2.2.).

The material from PCR 1 was amplified either in a standard PCR reaction (library 2.1) or in a second error-prone PCR reaction (PCR 2) consisting of a denaturing step at 94° C. for 2 min, 20 cycles of 94° C. for 30 s, 57° C. for 30 s and 72° C. for 60 s and finally elongation at 72° C. for 7 minutes with 0.3 mM of each primers in a 100 μl total volume with 0.2 mM dATP, 0.2 mM dGTP, 1 mM dTTP, 1 mM dCTP, 7 mM MgCl₂, 0.5 mM MnCl₂, and 1.25 U of AmpliTaq DNA polymerase (library 2.2). 5 PCR 2 reactions were performed per PCR 1 reaction.

### Results

TABLE 5

| | Mutations | New | Frequency | Number of | Frequency | Library |
|---|---|---|---|---|---|---|
| | in starting | mutations/ | recombined | recombinations/ | unique | size |
| Library¹ | material² | clone² | sequences (%) | recombined clone | sequences (%) | (clones) |
| 2.1 | 26 (18) | 0.2 | 28 | 1.4 | 81 | 2 × 10⁵ |
| 2.2 | 26 (18) | 2.4 | 48 | 1.6 | 97 | 7 × 10⁵ |

¹Sequence analysis was performed on 24 clones from each library.
²Mutations shown as base changes

1. A method for generating a polynucleotide sequence or population of sequences from parent polynucleotide sequence, the method comprising the steps of:
(a) providing a population of parent polynucleotide molecules, which population comprises plus and minus strands;
(b) treating the population of parent polynucleotide molecules to generate a population of polynucleotide fragments thereof;
(c) incubating the population of polynucleotide fragments under conditions which permit the formation of overlapping fragment pairs; and
(d) amplifying the overlapping fragment pairs using a polymerase to generate one or more product polynucleotide molecules which differ in sequence from the parent polynucleotide molecules
wherein step (d) is performed, at least in part, under conditions which favour the introduction of mutations into the one or more product polynucleotide molecules.

2. (canceled)

3. (canceled)

4. A method according to claim 1 wherein the parent polynucleotide molecules are double-stranded.

5. A method according to claim 1 wherein the parent polynucleotide molecules are single-stranded.

6. A method according to claim 1 wherein the population of parent polynucleotide molecules comprises a first sub-population and a second sub-population, wherein the first sub-population comprises or consists of single-stranded plus strands of the parent polynucleotide molecules and a second sub-population comprises or consists of single-stranded minus strands of the parent polynucleotide molecules.

7. (canceled)

8. A method according to claim 1 wherein the first sub-population and the second sub-population of parent polynucleotide molecules are treated separately in Step (b) and wherein at least one parameter of the reaction used for treating the first sub-population of polynucleotide molecules is different from the equivalent parameter used for treating the second sub-population of polynucleotide molecules.

9. (canceled)

10. A method according to claim 1 wherein step (b) comprises exposing the parent polynucleotide molecules to one or more nucleases.

11. (canceled)

12. A method according to claim 10 wherein the nuclease is an exonuclease.

13-17. (canceled)

18. A method according to claim 1 wherein step (c) comprises first incubating the population of polynucleotide fragments under conditions which permit denaturation of double-stranded fragments followed by incubating the population of polynucleotide fragments under conditions which permit re-annealing of single-stranded fragments to generate overlapping fragment pairs.

19. A method according to claim 1 wherein step (d) comprises repeated cycles of:
(i) extending the polynucleotide strands of the overlapping fragments pairs to generate extended fragment pairs;
(ii) denaturation of the constituent strands of the extended fragment pairs; and
(iii) re-annealing of the constituent strands to generate new overlapping fragment pairs.

20-22. (canceled)

23. A method according to claim 1 wherein, in step (d), the conditions which favour the introduction of mutations into the one or more product polynucleotide molecules permit error-prone polymerisation.

24. A method according to claim 23 wherein step (d) comprises the use of an error-prone polymerase to introduce mutations into the one or more product polynucleotide molecules.

25. (canceled)

26. A method according to claim 23 wherein step (d) comprises the use of error-prone PCR to introduce mutations into the one or more product polynucleotide molecules.

27. A method according to claim 23 wherein the error-prone polymerisation has an error rate of at least 0.01%, for example at least 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 2.0%, 2.5%, 3%, 3.5%, 4%, 4.5%, 5%, 10%, 20%, 30% or higher, and most preferably between 0.08% and 5%.

28. A method according to claim 23 wherein, in step (d), the conditions which favour the introduction of mutations into the one or more product polynucleotide molecules comprise the use of a mutagenesis enhancer in the polymerisation reaction.

29-31. (canceled)

32. A method according to claim 1 wherein, in step (d), the conditions which favour the introduction of mutations into the one or more product polynucleotide molecules comprise altering the concentration of one or more components in the in the polymerisation reaction.

33-39. (canceled)

40. A method according to claim 23 wherein, in step (d), the conditions which favour the introduction of mutations into the one or more product polynucleotide molecules comprise altering the temperature of the elongation step in the polymerisation reaction.

41. (canceled)

42. (canceled)

43. A method according to claim 1 wherein the mutation introduced into the one or more product polynucleotide molecules in step (d) is associated with an altered property of the encoded polypeptide.

44. A method according to claim 1 further comprising the step of expressing at least one of the product polynucleotide molecules generated in step (d) to produce the encoded polypeptide.

45-49. (canceled)

50. A method for making a polypeptide having altered properties, the method comprising the following steps:
(a) generating mutated forms of a parent polynucleotide using a method according to claim 1;
(b) expressing the variant polynucleotides produced in step (a) to produce variant polypeptides;
(c) screening the variant polypeptides for altered properties; and
(d) selecting a polypeptide having altered properties from the variant polypeptides.

51-54. (canceled)

55. A process for preparing a pharmaceutical composition which comprises, following the identification of a polynucleotide and/or encoded polypeptide with altered sequence or characteristics by a method according to claim 1, adding said polynucleotide and/or encoded polypeptide to a pharmaceutically acceptable carrier.

56. (canceled)

57. (canceled)


SEQUENCE LISTING (8 sequences)

SEQ ID NO: 1 - DNA, 44 bp, Artificial Sequence
  1736 SfiI forward primer
attactcgcggcccagccggccatggcccacaggtcaagctcga

SEQ ID NO: 2 - DNA, 35 bp, Artificial Sequence
  1735 NotI reversed primer
ttagagcctgcggccgccttgtcatcgtcgtcctt

SEQ ID NO: 3 - DNA, 44 bp, Artificial Sequence; identical to SEQ ID NO: 1
  1664 SfiI forward primer

SEQ ID NO: 4 - DNA, 35 bp, Artificial Sequence; identical to SEQ ID NO: 2
  1635 NotI reversed primer

SEQ ID NO: 5 - DNA, 38 bp, Artificial Sequence
  Error prone PCR forward primer
tcgcggcccagccggccatggcctttacttttgaaccg

SEQ ID NO: 6 - DNA, 35 bp, Artificial Sequence
  Error prone PCR reverse primer
gcctgcggccgcagatctaccattaattacataag

SEQ ID NO: 7 - protein, 39 aa, Staphylococcus aureus
NSGLPTTLGKLDERLRNYLKKGTKNSAQFEKMVILTENK

SEQ ID NO: 8 - protein, 44 aa, Staphylococcus aureus
GYYTVYLNTPLAEDRKNVELLGKMYKTYFFKKGESKSSYVINGP